(19) United States
(12) Patent Application Publication
(10) Pub. No.: US 2024/0342283 A1
(43) Pub. Date: Oct. 17, 2024

(54) ROR1 CAR or ROR1 / CD19 Dual CAR T Cells for the Treatment of Tumors

(71) Applicants: Shandong Boan Biotechnology Co., Ltd., Yantai (CN); Boan Boston LLC, Woburn, MA (US); NANJING BOAN BIOTECHNOLOGY CO. LTD., Nanjing, Jiangsu (CN)

(72) Inventors: Mengqi Zong, Yantai (CN); Jie Jiao, Yantai (CN); Jianxia Feng, Yantai (CN); Michael Harris, Yantai (CN); Xin Kai, Yantai (CN); Li Zhou, Yantai (CN); Ninghai Wang, Yantai (CN); Liangjun Wei, Yantai (CN); Hao Chen, Yantai (CN); Xian Wen, Yantai (CN); Changlin Dou, Yantai (CN); Chuangchuang Dong, Yantai (CN)

(21) Appl. No.: 18/682,673

(22) PCT Filed: Aug. 15, 2022
(86) PCT No.: PCT/CN2022/112449

§ 371 Date: Feb. 9, 2024

(30) Foreign Application Priority Data
Aug. 19, 2021 (WO) PCT/CN2021/113420
Apr. 29, 2022 (CN) 202210425699.0

(51) Int. Cl. A61K 39/00; A61P 35/00; C07K 14/705; C07K 14/725; C07K 16/28; C12N 5/0783

(52) U.S. Cl. CPC A61K 39/464462; A61K 39/4611; A61K 39/4631; A61K 39/464412; A61P 35/00; C07K 14/7051; C07K 14/70517; C07K 14/70521; C07K 14/70578; C07K 16/2803; C12N 5/0636; C07K 2317/24; C07K 2317/31; C07K 2317/622; C07K 2319/03; C12N 2510/00

(57) ABSTRACT

An ROR1 CAR or ROR1/CD19 Dual CAR for the treatment of tumors. The T cells expressing ROR1 CAR or ROR1/CD19 Dual CAR can be stimulated by ROR1-positive or ROR1/CD19-positive cells, and have cytotoxicity against ROR1-positive or ROR1/CD19-positive cells.

This application claims the benefit of International Application No. PCT/CN2021/113420, entitled “Stealth Chimeric Antigen Receptor and Use Thereof in Reducing Cytotoxicity towards Normal Cells”, filed on Aug. 19, 2021, and Chinese Patent Application No. 202210425699.0, entitled “ROR1 antibody or antigen-binding fragment thereof” filed on Apr. 29, 2022; the contents of which are herein incorporated by reference in their entireties.

### TECHNICAL FIELD

The present invention belongs to the field of biomedicine or biopharmaceuticals, particularly, to the treatment of tumors by cellular therapy, and more particularly, to the treatment of tumors with high expression of Receptor tyrosine kinase-like orphan receptor 1 (ROR1) or both ROR1 and CD19 using transgenic T lymphocytes expressing ROR1 CAR or ROR1/CD19 Dual CAR.

### BACKGROUND

Receptor tyrosine kinase-like orphan receptor 1 (ROR1) is a transmembrane protein within the ROR family, which consists of ROR1 and ROR2. Human ROR1/2 have 58% amino acid identity overall and 68% amino acid identity in the kinase domain. Amino acid sequence identity is highly conserved among different species within the ROR1 and ROR2 subgroups respectively. A 97% amino acid sequence identity between human and mouse ROR1 (hROR1 & mROR1) has been noted. Human ROR1 is located on chromosome 1 (1p31.3) with a protein size of 937 amino acids and molecular weight of approximately 105 kDa. The structure of human ROR1 consists of an extracellular immunoglobulin-like (Ig) domain at the amino-terminus, a Frizzled (Fz) domain, a kringle (Kr) domain, a transmembrane domain, a tyrosine kinase domain, a Serine/Threonine-rich domain (Ser/Thr), a proline-rich (PR) domain, and a second Ser/Thr domain at the carboxy-terminus. The Ig domain is at the far end of the extracellular part. The precise role of the Ig domain is unknown, but it may be involved in protein and ligand interactions as well as with interfering with the Fz and Kr domains. The Fz domain is similar to the Wnt binding domain of Frizzled receptors and is thought to mediate the interaction between ROR1 receptor and its ligands such as Wnt5a. The Kr domain is a highly-folded cysteine-rich domain located in close proximity to the plasma membrane, which is required for heterodimerization of ROR1 and ROR2.

While ROR1 expression is largely embryonal, there is widespread evidence to suggest that high expression levels of ROR1 are associated with both hematological malignancies and solid tumors. Strong expression of ROR1 was initially identified in B-Cell chronic lymphocytic leukemia (CLL), while completely absent in healthy peripheral blood mononuclear cells (PBMC). Further studies indicate both ROR1 and gene expression are upregulated in several additional hematological malignancies such as acute lymphocytic leukemia (ALL), mantle cell lymphoma (MCL), follicular lymphoma (FL), diffuse large B-cell lymphoma (DLBCL), marginal zone lymphoma (MZL), myelomas, and myeloid leukemias. Furthermore, there is a correlation between ROR1 expression and disease progression. A transcriptome analysis of 1568 CLL patients reveals that CLL cases that expressed a high level of ROR1 tend to have more aggressive disease progression and shorter overall survival time than patients with a low level of ROR1.

ROR1 expression has also been observed in various solid tumors. An immunohistochemistry (IHC) analysis of a variety of solid cancers revealed that ROR1 expression patterns varied from moderate to high depending on the type of cancer. Among the ROR1-positive samples were ovarian cancers (78/144), skin cancers (49/55), pancreatic cancers (45/57), colon cancers (63/110), lung cancers (52/58), adrenal cancers (10/12), uterine cancers (28/29), and testicular (35/48) and prostate cancers (19/21). In breast cancer, ROR1 was shown to be expressed in human neoplastic cells but absent in stromal cells. ROR1 overexpression in breast cancer was linked to aggressive disease. Breast cancer cell lines with strong ROR1 expression were more aggressive and invasive but declined in non-migrating cells. An IHC study of 232 lung adenocarcinoma (ADC) patients supported the identification of ROR1 expression as a clinicopathological feature of lung ADC. Those IHC analyses showed that 57.9% of lung ADC patients at stage III-IV exhibited high expression of ROR1 protein, whereas only 21.3% of patients at stage I-II showed high ROR1 expression. Moreover, survival analysis also indicated a linear relationship between high ROR1 expression and worse overall survival rates. Taken together, the present literature provides strong evidence to support the identification of ROR1 as a promising therapeutic target for anticancer therapy.

In recent years, many pharmaceutical companies have deployed ROR1-targeting drugs, including monoclonal antibodies, antibody-drug conjugates (ADCs), bispecific antibodies, CAR-T therapies, etc.

Also, Octenal Therapeutics' Phase ½ clinical study of ROR1 monoclonal antibody Zilovertamab (formerly called cirmtuzumab or UC-961) in combination with Ibrutinib for the treatment of relapsed/refractory cell lymphomas or primary/refractory chronic lymphocytic leukemia has yielded positive data; while VelosBio has disclosed Phase 1 clinical trial data for investigational drug VLS-101 (an ADC drug targeting ROR1) showing safety and antitumor efficacy.

Targeting of tumor antigens by CAR T cells causes selective pressure and downregulation of the tumor associated antigen in a process called antigen escape. During antigen escape, a second tumor associated antigen can be upregulated by the tumor cells, such as CD19. CD19 was also reported to be co-expressed with ROR1 in B-cell malignancies and other kinds of tumor cells. Dual targeting of both antigens is an effective way to prevent tumor relapse due to antigen escape. In cases where the tumor cells express multiple tumor-associated antigens, this dual targeting can be an effective way to enhance CAR efficacy.

How to provide a treatment for tumors with high expression of Receptor tyrosine kinase-like orphan receptor 1 (ROR1) or both Receptor tyrosine kinase-like orphan receptor 1 (ROR1) and CD19 has been recognized in the art as a problem to be solved.

### SUMMARY OF THE INVENTION

The present invention provides an ROR1 CAR or ROR1/CD19 Dual CAR for the treatment of tumors with high expression of Receptor tyrosine kinase-like orphan receptor 1 (ROR1) or both Receptor tyrosine kinase-like orphan receptor 1 (ROR1) and CD19.

In a first aspect, the present invention provides a chimeric antigen receptor (CAR) that binds to ROR1, wherein the CAR comprises a signal peptide, antibody or antigen-binding fragment thereof, hinge domain, transmembrane domain and/or intracellular domain.

In an embodiment, the signal peptide can be selected from CD8a signal peptide, VH3 signal peptide, IL2 signal peptide or the like; the hinge domain can be selected from CD8 hinge domain, a CD28 hinge domain or the like; the transmembrane domain can be selected from CD8a transmembrane domain, CD28 transmembrane domain, 4-1BB transmembrane domain or transmembrane-juxtamembrane domain or the like, and the transmembrane-juxtamembrane domain can be selected from Seizure 6-like protein 2 (SEZ6L2) transmembrane-juxtamembrane domain, or the like; and the intracellular domain can be selected from CD28 intracellular domain, 4-1BB intracellular domain, OX40 intracellular domain, CD3ζ intracellular domain or the like.

In a further embodiment, the signal peptide is CD8a signal peptide, the hinge domain is CD8 hinge domain, the transmembrane domain is CD8a transmembrane domain or Seizure 6-like protein 2 (SEZ6L2) transmembrane-juxtamembrane domain, and the intracellular domain is 4-1BB intracellular domain and/or CD3ζ intracellular domain.

In one embodiment, the antibody or antigen-binding fragment is an ROR1; preferably, VH and VL of the scFv are linked through a linker; preferably, through a (GGGGS)₃ or (GGGGSGGGGSGGGGS) linker; preferably, in the order of VH-(GGGGS)₃-VL from N terminus to C terminus.

In a specific embodiment, the present invention provides a chimeric antigen receptor (CAR) comprising,
(1) an extracellular ligand-binding domain comprising scFv specifically binding to Receptor tyrosine kinase-like Orphan Receptor 1 (ROR1);
(2) a transmembrane domain; wherein preferably, the transmembrane domain is CD8 transmembrane domain; or
a transmembrane (tm) linking juxtamembrane jm) domain, wherein the transmembrane linking juxtamembrane domain comprises a Seizure 6-like Protein 2 (SEZ6L2) transmembrane domain and a SEZ6L2 juxtamembrane domain; and
(3) an intracellular domain; wherein preferably, the intracellular domain comprises a signaling domain; more preferably, the signaling domain comprises one or more signaling domains selected from the group consisting of a 4-1BB signaling domain, a CD28 signaling domain and a CD3ζ signaling domain;
wherein the scFv specifically binding to ROR1 comprises:
HCDR1 shown in SEQ ID NO.: 10, HCDR2 shown in SEQ ID NO.: 11, HCDR3 shown in SEQ ID NO.: 12, LCDR1 shown in SEQ ID NO.: 27, LCDR2 shown in SEQ ID NO.: 28 and LCDR3 shown in SEQ ID NO.: 29;
HCDR1 shown in SEQ ID NO.: 1, HCDR2 shown in SEQ ID NO.: 2, HCDR3 shown in SEQ ID NO.: 3, LCDR1 shown in SEQ ID NO.: 18, LCDR2 shown in SEQ ID NO.: 19 and LCDR3 shown in SEQ ID NO.: 20;
HCDR1 shown in SEQ ID NO.: 4, HCDR2 shown in SEQ ID NO.: 5, HCDR3 shown in SEQ ID NO.: 6, LCDR1 shown in SEQ ID NO.: 21, LCDR2 shown in SEQ ID NO.: 22 and LCDR3 shown in SEQ ID NO.: 23;
HCDR1 shown in SEQ ID NO.: 7, HCDR2 shown in SEQ ID NO.: 8, HCDR3 shown in SEQ ID NO.: 9, LCDR1 shown in SEQ ID NO.: 24, LCDR2 shown in SEQ ID NO.: 25 and LCDR3 shown in SEQ ID NO.: 26;
HCDR1 shown in SEQ ID NO.: 10, HCDR2 shown in SEQ ID NO.: 11, HCDR3 shown in SEQ ID NO.: 12, LCDR1 shown in SEQ ID NO.: 27, LCDR2 shown in SEQ ID NO.: 28 and LCDR3 shown in SEQ ID NO.: 29;
HCDR1 shown in SEQ ID NO.: 13, HCDR2 shown in SEQ ID NO.: 14, HCDR3 shown in SEQ ID NO.: 15, LCDR1 shown in SEQ ID NO.: 30, LCDR2 shown in SEQ ID NO.: 31 and LCDR3 shown in SEQ ID NO.: 32;
HCDR1 shown in SEQ ID NO.: 10, HCDR2 shown in SEQ ID NO.:16, HCDR3 shown in SEQ ID NO.: 17, LCDR1 shown in SEQ ID NO.: 33, LCDR2 shown in SEQ ID NO.: 34 and LCDR3 shown in SEQ ID NO.: 35;
HCDR1 shown in SEQ ID NO.: 10, HCDR2 shown in SEQ ID NO.:11, HCDR3 shown in SEQ ID NO.: 12, LCDR1 shown in SEQ ID NO.: 27, LCDR2 shown in SEQ ID NO.: 28 and LCDR3 shown in SEQ ID NO.: 29; or
HCDR1 shown in SEQ ID NO.: 83, HCDR2 shown in SEQ ID NO.:84, HCDR3 shown in SEQ ID NO.: 85, LCDR1 shown in SEQ ID NO.:86, LCDR2 shown in SEQ ID NO.: 87 and LCDR3 shown in SEQ ID NO.: 88.

In an embodiment, the scFv specifically binding to ROR1 comprises:
VH comprising an amino acid sequence that is at least about 95%, 96%, 97%, 98%, 99% or 100% identical to the amino acid sequence represented by SEQ ID NO.: 57 and VL comprising an amino acid sequence that is at least about 95%, 96%, 97%, 98%, 99% or 100% identical to the amino acid sequence represented by SEQ ID NO.: 59;
VH comprising an amino acid sequence that is at least about 95%, 96%, 97%, 98%, 99% or 100% identical to the amino acid sequence represented by SEQ ID NO.: 44 and VL comprising an amino acid sequence that is at least about 95%, 96%, 97%, 98%, 99% or 100% identical to the amino acid sequence represented by SEQ ID NO.: 50;
VH comprising an amino acid sequence that is at least about 95%, 96%, 97%, 98%, 99% or 100% identical to the amino acid sequence represented by SEQ ID NO.: 45 and VL comprising an amino acid sequence that is at least about 95%, 96%, 97%, 98%, 99% or 100% identical to the amino acid sequence represented by SEQ ID NO.:51;
VH comprising an amino acid sequence that is at least about 95%, 96%, 97%, 98%, 99% or 100% identical to the amino acid sequence represented by SEQ ID NO.: 46 and VL comprising an amino acid sequence that is at least about 95%, 96%, 97%, 98%, 99% or 100% identical to the amino acid sequence represented by SEQ ID NO.:52;
VH comprising an amino acid sequence that is at least about 95%, 96%, 97%, 98%, 99% or 100% identical to the amino acid sequence represented by SEQ ID NO.:47 and VL comprising an amino acid sequence that is at least about 95%, 96%, 97%, 98%, 99% or 100% identical to the amino acid sequence represented by SEQ ID NO.:53;
VH comprising an amino acid sequence that is at least about 95%, 96%, 97%, 98%, 99% or 100% identical to the amino acid sequence represented by SEQ ID NO.: 48 and VL comprising an amino acid sequence that is at least about 95%, 96%, 97%, 98%, 99% or 100% identical to the amino acid sequence represented by SEQ ID NO.: 54;
VH comprising an amino acid sequence that is at least about 95%, 96%, 97%, 98%, 99% or 100% identical to the amino acid sequence represented by SEQ ID NO.: 49 and VL comprising an amino acid sequence that is at least about 95%, 96%, 97%, 98%, 99% or 100% identical to the amino acid sequence represented by SEQ ID NO.:55;
VH comprising an amino acid sequence that is at least about 95%, 96%, 97%, 98%, 99% or 100% identical to the amino acid sequence represented by SEQ ID NO.:56 and VL comprising an amino acid sequence that is at least about 95%, 96%, 97%, 98%, 99% or 100% identical to the amino acid sequence represented by SEQ ID NO.:59;
VH comprising an amino acid sequence that is at least about 95%, 96%, 97%, 98%, 99% or 100% identical to the amino acid sequence represented by SEQ ID NO.:57 and VL comprising an amino acid sequence that is at least about 95%, 96%, 97%, 98%, 99% or 100% identical to the amino acid sequence represented by SEQ ID NO.:58; or
VH comprising an amino acid sequence that is at least about 95%, 96%, 97%, 98%, 99% or 100% identical to the amino acid sequence represented by SEQ ID NO.:81 and VL comprising an amino acid sequence that is at least about 95%, 96%, 97%, 98%, 99% or 100% identical to the amino acid sequence represented by SEQ ID NO.:82.

In a further embodiment, the CAR comprises from N-terminal to C-terminal:
1) ROR1 scFv-CD8Hinge-CD8 tm-4-1BB-CD3ζ; or
2) ROR1 scFv-CD8Hinge-SEZ6L2 tm jm-CD3ζ;
wherein preferably, the N-terminal of the CAR further contains a leader sequence.

In a further embodiment, the leader sequence comprises an amino acid sequence that is at least about 95%, 96%, 97%, 98%, 99% or 100% identical to the amino acid sequence represented by SEQ ID NO. 61.

In a further embodiment, the CD8 Hinge comprises an amino acid sequence that is at least about 95%, 96%, 97%, 98%, 99% or 100% identical to the amino acid sequence represented by SEQ ID NO.: 62.

In a further embodiment, the CD8tm comprises an amino acid sequence that is at least about 95%, 96%, 97%, 98%, 99% or 100% identical to the amino acid sequence represented by SEQ ID NO.: 63.

In a further embodiment, the 4-1BB comprises an amino acid sequence that is at least about 95%, 96%, 97%, 98%, 99% or 100% identical to the amino acid sequence represented by SEQ ID NO.: 64.

In a further embodiment, the CD3ζ intracellular domain comprises an amino acid sequence that is at least about 95%, 96%, 97%, 98%, 99% or 100% identical to the amino acid sequence represented by SEQ ID NO.: 65.

In a further embodiment, the SEZ6L2 transmembrane-juxtamembrane domain comprises an amino acid sequence that is at least about 95%, 96%, 97%, 98%, 99% or 100% identical to the amino acid sequence represented by SEQ ID NO.: 66.

In a further embodiment, wherein the CAR comprises from N-terminal to C-terminal: 1) ROR1 scFv-CD8Hinge-CD8 tm-4-1BB-CD3ζ; or
2) ROR1 scFv-CD8Hinge-SEZ6L2 tm jm-CD3ζ;
the N-terminal of the CAR further contains a leader sequence, wherein the leader sequence comprises an amino acid sequence that is at least about 95%, 96%, 97%, 98%, 99% or 100% identical to the amino acid sequence represented by SEQ ID NO. 61,
the CD8 Hinge comprises an amino acid sequence that is at least about 95%, 96%, 97%, 98%, 99% or 100% identical to the amino acid sequence represented by SEQ ID NO.: 62,
the CD8 tm comprises an amino acid sequence that is at least about 95%, 96%, 97%, 98%, 99% or 100% identical to the amino acid sequence represented by SEQ ID NO.: 63,
the 4-1BB comprises an amino acid sequence that is at least about 95%, 96%, 97%, 98%, 99% or 100% identical to the amino acid sequence represented by SEQ ID NO.: 64,
the CD3ζ intracellular domain comprises an amino acid sequence that is at least about 95%, 96%, 97%, 98%, 99% or 100% identical to the amino acid sequence represented by SEQ ID NO.: 65, and
the SEZ6L2 transmembrane-juxtamembrane domain (SEZ6L2 tm jm) comprises an amino acid sequence that is at least about 95%, 96%, 97%, 98%, 99% or 100% identical to the amino acid sequence represented by SEQ ID NO.: 66.

In a preferred embodiment, the ROR1 CAR comprises, from N-terminal to C-terminal:
1) ROR1 scFv-CD8Hinge-CD8 tm-4-1BB-CD3ζ; or
2) ROR1 scFv-CD8Hinge-SEZ6L2 tm jm-CD3ζ;
wherein the ROR1 scFv comprises: HCDR1 shown in SEQ ID NO.: 10, HCDR2 shown in SEQ ID NO.: 11, HCDR3 shown in SEQ ID NO.: 12, LCDR1 shown in SEQ ID NO.: 27, LCDR2 shown in SEQ ID NO.: 28 and LCDR3 shown in SEQ ID NO.: 29;
wherein preferably, the ROR1 scFv comprises: VH shown in SEQ ID NO.: 57 and VL shown in SEQ ID NO.: 59; wherein
the ROR1 CAR further contains a leader sequence, wherein the leader sequence comprises an amino acid sequence that is at least about 95%, 96%, 97%, 98%, 99% or 100% identical to the amino acid sequence represented by SEQ ID NO. 61,
the CD8 Hinge comprises an amino acid sequence that is at least about 95%, 96%, 97%, 98%, 99% or 100% identical to the amino acid sequence represented by SEQ ID NO.: 62,
the CD8 tm comprises an amino acid sequence that is at least about 95%, 96%, 97%, 98%, 99% or 100% identical to the amino acid sequence represented by SEQ ID NO.: 63,
the 4-1BB comprises an amino acid sequence that is at least about 95%, 96%, 97%, 98%, 99% or 100% identical to the amino acid sequence represented by SEQ ID NO.: 64,
the CD3ζ intracellular domain comprises an amino acid sequence that is at least about 95%, 96%, 97%, 98%, 99% or 100% identical to the amino acid sequence represented by SEQ ID NO.: 65, and
the SEZ6L2 transmembrane-juxtamembrane domain (SEZ6L2 tm jm) comprises an amino acid sequence that is at least about 95%, 96%, 97%, 98%, 99% or 100% identical to the amino acid sequence represented by SEQ ID NO.: 66.

In a specific embodiment, the present invention provides a chimeric antigen receptor (CAR) comprising,
(1) an extracellular ligand-binding domain comprising scFv specifically binding to CD19;
(2) a transmembrane domain; wherein preferably, the transmembrane domain is CD8 transmembrane domain; or
a transmembrane (tm) linking juxtamembrane (jm) domain, wherein the transmembrane linking juxtamembrane domain comprises a Seizure 6-like Protein 2 (SEZ6L2) transmembrane domain and a SEZ6L2 juxtamembrane domain;
(3) an intracellular domain; wherein preferably, the intracellular domain comprises signaling domain; more preferably, the signaling domain comprises one or more signaling domains selected from the group consisting of a 4-1BB signaling domain, a CD28 signaling domain and a CD3ζ signaling domain;
wherein the scFv specifically binding to CD19 comprises:
HCDR1 shown in SEQ ID NO.: 37, HCDR2 shown in SEQ ID NO.: 38, HCDR3 shown in SEQ ID NO.: 39, LCDR1 shown in SEQ ID NO.: 41, LCDR2 shown in SEQ ID NO.: 42 and LCDR3 shown in SEQ ID NO.: 43; or
HCDR1 shown in SEQ ID NO.: 37, HCDR2 shown in SEQ ID NO.: 38, HCDR3 shown in SEQ ID NO.: 40, LCDR1 shown in SEQ ID NO.: 41, LCDR2 shown in SEQ ID NO.: 42 and LCDR3 shown in SEQ ID NO.: 43.

In an embodiment, the scFv specifically binding to CD19 comprises:
VH comprising an amino acid sequence that is at least about 95%, 96%, 97%, 98%, 99% or 100% identical to the amino acid sequence represented by SEQ ID NO.: 69 and VL comprising an amino acid sequence that is at least about 95%, 96%, 97%, 98%, 99% or 100% identical to the amino acid sequence represented by SEQ ID NO.: 70;
VH comprising an amino acid sequence that is at least about 95%, 96%, 97%, 98%, 99% or 100% identical to the amino acid sequence represented by SEQ ID NO.: 71 and VL comprising an amino acid sequence that is at least about 95%, 96%, 97%, 98%, 99% or 100% identical to the amino acid sequence represented by SEQ ID NO.: 75;
VH comprising an amino acid sequence that is at least about 95%, 96%, 97%, 98%, 99% or 100% identical to the amino acid sequence represented by SEQ ID NO.: 71 and VL comprising an amino acid sequence that is at least about 95%, 96%, 97%, 98%, 99% or 100% identical to the amino acid sequence represented by SEQ ID NO.:76;
VH comprising an amino acid sequence that is at least about 95%, 96%, 97%, 98%, 99% or 100% identical to the amino acid sequence represented by SEQ ID NO.: 71 and VL comprising an amino acid sequence that is at least about 95%, 96%, 97%, 98%, 99% or 100% identical to the amino acid sequence represented by SEQ ID NO.:77;
VH comprising an amino acid sequence that is at least about 95%, 96%, 97%, 98%, 99% or 100% identical to the amino acid sequence represented by SEQ ID NO.:71 and VL comprising an amino acid sequence that is at least about 95%, 96%, 97%, 98%, 99% or 100% identical to the amino acid sequence represented by SEQ ID NO.:78;
VH comprising an amino acid sequence that is at least about 95%, 96%, 97%, 98%, 99% or 100% identical to the amino acid sequence represented by SEQ ID NO.: 72 and VL comprising an amino acid sequence that is at least about 95%, 96%, 97%, 98%, 99% or 100% identical to the amino acid sequence represented by SEQ ID NO.: 75;
VH comprising an amino acid sequence that is at least about 95%, 96%, 97%, 98%, 99% or 100% identical to the amino acid sequence represented by SEQ ID NO.:72 and VL comprising an amino acid sequence that is at least about 95%, 96%, 97%, 98%, 99% or 100% identical to the amino acid sequence represented by SEQ ID NO.:76;
VH comprising an amino acid sequence that is at least about 95%, 96%, 97%, 98%, 99% or 100% identical to the amino acid sequence represented by SEQ ID NO.:72 and VL comprising an amino acid sequence that is at least about 95%, 96%, 97%, 98%, 99% or 100% identical to the amino acid sequence represented by SEQ ID NO.:77;
VH comprising an amino acid sequence that is at least about 95%, 96%, 97%, 98%, 99% or 100% identical to the amino acid sequence represented by SEQ ID NO.:72 and VL comprising an amino acid sequence that is at least about 95%, 96%, 97%, 98%, 99% or 100% identical to the amino acid sequence represented by SEQ ID NO.:78;
VH comprising an amino acid sequence that is at least about 95%, 96%, 97%, 98%, 99% or 100% identical to the amino acid sequence represented by SEQ ID NO.:73 and VL comprising an amino acid sequence that is at least about 95%, 96%, 97%, 98%, 99% or 100% identical to the amino acid sequence represented by SEQ ID NO.:75;
VH comprising an amino acid sequence that is at least about 95%, 96%, 97%, 98%, 99% or 100% identical to the amino acid sequence represented by SEQ ID NO.:73 and VL comprising an amino acid sequence that is at least about 95%, 96%, 97%, 98%, 99% or 100% identical to the amino acid sequence represented by SEQ ID NO.:76;
VH comprising an amino acid sequence that is at least about 95%, 96%, 97%, 98%, 99% or 100% identical to the amino acid sequence represented by SEQ ID NO.:73 and VL comprising an amino acid sequence that is at least about 95%, 96%, 97%, 98%, 99% or 100% identical to the amino acid sequence represented by SEQ ID NO.:77;
VH comprising an amino acid sequence that is at least about 95%, 96%, 97%, 98%, 99% or 100% identical to the amino acid sequence represented by SEQ ID NO.:73 and VL comprising an amino acid sequence that is at least about 95%, 96%, 97%, 98%, 99% or 100% identical to the amino acid sequence represented by SEQ ID NO.:78;
VH comprising an amino acid sequence that is at least about 95%, 96%, 97%, 98%, 99% or 100% identical to the amino acid sequence represented by SEQ ID NO.:74 and VL comprising an amino acid sequence that is at least about 95%, 96%, 97%, 98%, 99% or 100% identical to the amino acid sequence represented by SEQ ID NO.:75;
VH comprising an amino acid sequence that is at least about 95%, 96%, 97%, 98%, 99% or 100% identical to the amino acid sequence represented by SEQ ID NO.:74 and VL comprising an amino acid sequence that is at least about 95%, 96%, 97%, 98%, 99% or 100% identical to the amino acid sequence represented by SEQ ID NO.:76;
VH comprising an amino acid sequence that is at least about 95%, 96%, 97%, 98%, 99% or 100% identical to the amino acid sequence represented by SEQ ID NO.:74 and VL comprising an amino acid sequence that is at least about 95%, 96%, 97%, 98%, 99% or 100% identical to the amino acid sequence represented by SEQ ID NO.:77; or
VH comprising an amino acid sequence that is at least about 95%, 96%, 97%, 98%, 99% or 100% identical to the amino acid sequence represented by SEQ ID NO.:74 and VL comprising an amino acid sequence that is at least about 95%, 96%, 97%, 98%, 99% or 100% identical to the amino acid sequence represented by SEQ ID NO.:78.

In a further embodiment, the CAR comprises from N-terminal to C-terminal:
1) CD19 scFv-CD8Hinge-CD8 tm-4-1BB-CD3Q; or
2) CD19 scFv-CD8Hinge-SEZ6L2 tm jm-CD3ζ;
wherein preferably, the N-terminal of the CAR further contains a leader sequence.

In a further embodiment, the leader sequence comprises an amino acid sequence that is at least about 95%, 96%, 97%, 98%, 99% or 100% identical to the amino acid sequence represented by SEQ ID NO. 61.

In a further embodiment, the CD8 Hinge comprises an amino acid sequence that is at least about 95%, 96%, 97%, 98%, 99% or 100% identical to the amino acid sequence represented by SEQ ID NO.: 62.

In a further embodiment, the CD8tm comprises an amino acid sequence that is at least about 95%, 96%, 97%, 98%, 99% or 100% identical to the amino acid sequence represented by SEQ ID NO.: 63.

In a further embodiment, the 4-1BB comprises an amino acid sequence that is at least about 95%, 96%, 97%, 98%, 99% or 100% identical to the amino acid sequence represented by SEQ ID NO.: 64.

In a further embodiment, the CD3ζ intracellular domain comprises an amino acid sequence that is at least about 95%, 96%, 97%, 98%, 99% or 100% identical to the amino acid sequence represented by SEQ ID NO.: 65.

In a further embodiment, the SEZ6L2 transmembrane-juxtamembrane domain comprises an amino acid sequence that is at least about 95%, 96%, 97%, 98%, 99% or 100% identical to the amino acid sequence represented by SEQ ID NO.: 66.

In a further embodiment, the CAR comprises from N-terminal to C-terminal:
1) CD19 scFv-CD8Hinge-CD8 tm-4-1BB-CD3Q; or
2) CD19 scFv-CD8Hinge-SEZ6L2 tm jm-CD3ζ;
wherein the N-terminal of the CAR further contains a leader sequence, wherein the leader sequence comprises an amino acid sequence that is at least about 95%, 96%, 97%, 98%, 99% or 100% identical to the amino acid sequence represented by SEQ ID NO. 61,
the CD8 Hinge comprises an amino acid sequence that is at least about 95%, 96%, 97%, 98%, 99% or 100% identical to the amino acid sequence represented by SEQ ID NO.: 62,
the CD8 tm comprises an amino acid sequence that is at least about 95%, 96%, 97%, 98%, 99% or 100% identical to the amino acid sequence represented by SEQ ID NO.: 63,
the 4-1BB comprises an amino acid sequence that is at least about 95%, 96%, 97%, 98%, 99% or 100% identical to the amino acid sequence represented by SEQ ID NO.: 64,
the CD3ζ intracellular domain comprises an amino acid sequence that is at least about 95%, 96%, 97%, 98%, 99% or 100% identical to the amino acid sequence represented by SEQ ID NO.: 65, and
the SEZ6L2 transmembrane-juxtamembrane domain (SEZ6L2 tm jm) comprises an amino acid sequence that is at least about 95%, 96%, 97%, 98%, 99% or 100% identical to the amino acid sequence represented by SEQ ID NO.: 66.

In a preferred embodiment, the CD19 CAR comprises, from N-terminal to C-terminal:
1) CD19 scFv-CD8Hinge-CD8 tm-4-1BB-CD3Q; or
2) CD19 scFv-CD8Hinge-SEZ6L2 tm jm-CD3ζ; and the CD19 scFv comprises: HCDR1 shown in SEQ ID NO.: 37, HCDR2 shown in SEQ ID NO.: 38, HCDR3 shown in SEQ ID NO.: 39, LCDR1 shown in SEQ ID NO.: 41, LCDR2 shown in SEQ ID NO.: 42 and LCDR3 shown in SEQ ID NO.: 43; wherein
preferably, the CD19 scFv comprises: VH shown in SEQ ID NO.: 69 and VL shown in SEQ ID NO.: 70; and
preferably, the SEZ6L2 transmembrane-juxtamembrane domain (SEZ6L2 tm jm) is shown in SEQ ID NO.: 66.

In a second aspect, the present invention provides a dual CAR comprising: a first CAR, and
a second CAR comprising:
(1) an extracellular ligand-binding domain comprising scFv specifically binding to a predetermined antigen; wherein the predetermined antigen is a tumor-associated antigen (TAA); more preferably, the TAA is selected from one or more of: CEA, Claudin 18.2, CGC3, CD38, CD19, CD20, CD22, BCMA, CAIX, CD446, CD13, EGFR, EGFRvIII, EpCam, GD2, EphA2, HER1, HER2, ICAM-1, IL13Ra2, Mesothelin, MUC1, MUC16, PSCA, NY-ESO-1, MART-1, WT1, MAGE-A10, MAGE-A3, MAGE-A4, EBV, NKG2D, PD1, PD-L1, CD25, IL-2 and CD3;
(2) a transmembrane domain, wherein preferably, the transmembrane domain is CD8 transmembrane domain; or
a transmembrane (tm) linking juxtamembrane jm) domain, wherein the transmembrane linking juxtamembrane domain comprises a Seizure 6-like Protein 2 (SEZ6L2) transmembrane domain and a SEZ6L2 juxtamembrane domain;
(3) an intracellular domain; wherein preferably, the intracellular domain comprises a signaling domain; more preferably, the signaling domain comprises one or more signaling domains selected from the group consisting of a 4-1BB signaling domain, a CD28 signaling domain and a CD3ζ signaling domain;
preferably, the first CAR targets ROR1 and the second CAR targets another antigen,
preferably, the first CAR and the second CAR are linked by P2A.

In an embodiment, the TAA is CD19, and the CD19 scFv comprises:
HCDR1 shown in SEQ ID NO.: 37, HCDR2 shown in SEQ ID NO.: 38, HCDR3 shown in SEQ ID NO.: 39, LCDR1 shown in SEQ ID NO.: 41, LCDR2 shown in SEQ ID NO.: 42 and LCDR3 shown in SEQ ID NO.: 43; or
HCDR1 shown in SEQ ID NO.: 37, HCDR2 shown in SEQ ID NO.: 38, HCDR3 shown in SEQ ID NO.: 40, LCDR1 shown in SEQ ID NO.: 41, LCDR2 shown in SEQ ID NO.: 42 and LCDR3 shown in SEQ ID NO.: 43.

In a further embodiment, the CD19 scFv comprises:
VH shown in SEQ ID NO.: 69 and VL shown in SEQ ID NO.: 70;
VH shown in SEQ ID NO.: 71 and VL shown in SEQ ID NO.: 75;
VH shown in SEQ ID NO.: 71 and VL shown in SEQ ID NO.: 76;
VH shown in SEQ ID NO.: 71 and VL shown in SEQ ID NO.: 77;
VH shown in SEQ ID NO.: 71 and VL shown in SEQ ID NO.: 78;
VH shown in SEQ ID NO.: 72 and VL shown in SEQ ID NO.: 75;
VH shown in SEQ ID NO.: 72 and VL shown in SEQ ID NO.: 76;
VH shown in SEQ ID NO.: 72 and VL shown in SEQ ID NO.: 77;
VH shown in SEQ ID NO.: 72 and VL shown in SEQ ID NO.: 78;
VH shown in SEQ ID NO.: 73 and VL shown in SEQ ID NO.: 75;
VH shown in SEQ ID NO.: 73 and VL shown in SEQ ID NO.: 76;
VH shown in SEQ ID NO.: 73 and VL shown in SEQ ID NO.: 77;
VH shown in SEQ ID NO.: 73 and VL shown in SEQ ID NO.: 78;
VH shown in SEQ ID NO.: 74 and VL shown in SEQ ID NO.: 75;
VH shown in SEQ ID NO.: 74 and VL shown in SEQ ID NO.: 76;
VH shown in SEQ ID NO.: 74 and VL shown in SEQ ID NO.: 77; or
VH shown in SEQ ID NO.: 74 and VL shown in SEQ ID NO.: 78.

In a further embodiment, the dual CAR comprises, from N-terminal to C-terminal:
TAA scFv-CD8Hinge-CD8tm-4-1BB-CD3ζ-P2A-ROR1scFv-CD8Hinge-SEZ6L2 tm jm-CD3ζ; or
ROR1scFv-CD8Hinge-CD8tm-4-1BB-CD3Q-P2A-TAA scFv-CD8Hinge-SEZ6L2 tm jm-CD3ζ; wherein
preferably, the N-terminal of the CAR further contains a leader sequence; and
preferably, the C-terminal of the CAR further contains a P2A-EGFP sequence.

In a further embodiment, the leader sequence comprises an amino acid sequence that is at least about 95%, 96%, 97%, 98%, 99% or 100% identical to the amino acid sequence represented by SEQ ID NO. 61.

In a further embodiment, the CD8 Hinge comprises an amino acid sequence that is at least about 95%, 96%, 97%, 98%, 99% or 100% identical to the amino acid sequence represented by SEQ ID NO.: 62.

In a further embodiment, the CD8tm comprises an amino acid sequence that is at least about 95%, 96%, 97%, 98%, 99% or 100% identical to the amino acid sequence represented by SEQ ID NO.: 63.

In a further embodiment, the 4-1BB comprises an amino acid sequence that is at least about 95%, 96%, 97%, 98%, 99% or 100% identical to the amino acid sequence represented by SEQ ID NO.: 64.

In a further embodiment, the CD3ζ intracellular domain comprises an amino acid sequence that is at least about 95%, 96%, 97%, 98%, 99% or 100% identical to the amino acid sequence represented by SEQ ID NO.: 65.

In a further embodiment, the SEZ6L2 transmembrane-juxtamembrane domain comprises an amino acid sequence that is at least about 95%, 96%, 97%, 98%, 99% or 100% identical to the amino acid sequence represented by SEQ ID NO.: 66.

In a further embodiment, the EGFP comprises an amino acid sequence that is at least about 95%, 96%, 97%, 98%, 99% or 100% identical to the amino acid sequence represented by SEQ ID NO.: 67.

In a further embodiment, the P2A comprises an amino acid sequence that is at least about 95%, 96%, 97%, 98%, 99% or 100% identical to the amino acid sequence represented by SEQ ID NO.: 68.

In a further embodiment, the dual CAR comprises, from N-terminal to C-terminal:
TAA scFv-CD8Hinge-CD8tm-4-1BB-CD3ζ-P2A-ROR1scFv-CD8Hinge-SEZ6L2 tm jm-CD3ζ; or
ROR1scFv-CD8Hinge-CD8tm-4-1BB-CD3Q-P2A-TAA scFv-CD8Hinge-SEZ6L2 tm jm-CD3ζ; wherein
the N-terminal of the CAR further contains a leader sequence, and the C-terminal of the CAR further contains a P2A-EGFP sequence, wherein,
the leader sequence comprises an amino acid sequence that is at least about 95%, 96%, 97%, 98%, 99% or 100% identical to the amino acid sequence represented by SEQ ID NO. 61,
the CD8Hinge comprises an amino acid sequence that is at least about 95%, 96%, 97%, 98%, 99% or 100% identical to the amino acid sequence represented by SEQ ID NO.: 62,
the CD8tm comprises an amino acid sequence that is at least about 95%, 96%, 97%, 98%, 99% or 100% identical to the amino acid sequence represented by SEQ ID NO.: 63,
the 4-1BB intracellular domain comprises an amino acid sequence that is at least about 95%, 96%, 97%, 98%, 99% or 100% identical to the amino acid sequence represented by SEQ ID NO.: 64,
the CD3ζ intracellular domain comprises an amino acid sequence that is at least about 95%, 96%, 97%, 98%, 99% or 100% identical to the amino acid sequence represented by SEQ ID NO.: 65,
the SEZ6L2 transmembrane-juxtamembrane domain (SEZ6L2 tm jm) comprises an amino acid sequence that is at least about 95%, 96%, 97%, 98%, 99% or 100% identical to the amino acid sequence represented by SEQ ID NO.: 66,
the EGFP comprises an amino acid sequence that is at least about 95%, 96%, 97%, 98%, 99% or 100% identical to the amino acid sequence represented by SEQ ID NO.: 67, and
the P2A comprises an amino acid sequence that is at least about 95%, 96%, 97%, 98%, 99% or 100% identical to the amino acid sequence represented by SEQ ID NO. 68.

In a preferred embodiment, the dual CAR comprises, from N-terminal to C-terminal:
CD19 scFv-CD8Hinge-CD8tm-4-1BB-CD3Q-P2A-ROR1scFv-CD8Hinge-SEZ6L2 tm jm-CD3ζ; or
ROR1scFv-CD8Hinge-CD8tm-4-1BB-CD3ζ-P2A-CD19 scFv-CD8Hinge-SEZ6L2 tm jm-CD3ζ;
wherein the ROR1 scFv comprises: HCDR1 shown in SEQ ID NO.: 10, HCDR2 shown in SEQ ID NO.: 11, HCDR3 shown in SEQ ID NO.: 12, LCDR1 shown in SEQ ID NO.: 27, LCDR2 shown in SEQ ID NO.: 28 and LCDR3 shown in SEQ ID NO.: 29; wherein preferably, the ROR1 scFv comprises: VH shown in SEQ ID NO.: 57 and VL shown in SEQ ID NO.: 59;
the CD19 scFv comprises: HCDR1 shown in SEQ ID NO.: 37, HCDR2 shown in SEQ ID NO.: 38, HCDR3 shown in SEQ ID NO.: 39, LCDR1 shown in SEQ ID NO.: 41, LCDR2 shown in SEQ ID NO.: 42 and LCDR3 shown in SEQ ID NO.: 43; wherein preferably, the CD19 scFv comprises: VH shown in SEQ ID NO.: 69 and VL shown in SEQ ID NO.: 70;
wherein preferably, the SEZ6L2 transmembrane-juxtamembrane domain (SEZ6L2 tm jm) is shown in SEQ ID NO.: 66.

In a third aspect, the present invention provides a nucleic acid comprising a polynucleotide encoding the above-mentioned CAR or dual CAR.

In a fourth aspect, the present invention provides a vector comprising a polynucleotide encoding the above-mentioned CAR or dual CAR, or the above-mentioned nucleic acid. Preferably, the vector may be a viral vector; preferably, the viral vector includes, but is not limited to, a lentivirus vector, an adenovirus vector, an adeno-associated virus vector or a retrovirus vector; preferably, the vector may be a non-viral vector; preferably, the non-viral vector may be a transposon vector; preferably, the transposon vector may be a Sleeping Beauty vector, a PiggyBac vector, or the like; preferably, the vector may be a mammalian expression vector; preferably, the expression vector may be a bacterial expression vector; preferably, the expression vector may be a fungal expression vector.

In a fifth aspect, the present invention provides a cell comprising the CAR or dual CAR, or the nucleic acid or the vector according to any of the preceding aspects. The present invention also provides a cell that can express the CAR or dual CAR according to any of the preceding aspects. Preferably, the cell is a bacterial cell; preferably, the bacterial cell is an Escherichia coli cell or the like; preferably, the cell is a fungal cell; preferably, the fungal cell is a yeast cell; preferably, the yeast cell is a Pichia pastoris cell or the like; preferably, the cell is a mammalian cell; and preferably, the mammalian cell is a Chinese hamster ovary (CHO) cell, a human embryonic kidney cell (293), a stem cell, a B cell, a T cell, a DC cell, a NK cell, or the like. The present invention provides a CAR-T cell that comprises the nucleic acid or the vector according to any of the preceding aspects. The present invention also provides a CAR-T cell that can express the antibody or the antigen-binding fragment thereof, or the chimeric antigen receptor according to any of the preceding aspects.

In a sixth aspect, the present invention provides a composition comprising the CAR or dual CAR, the nucleic acid or the vector, or the cell according to any of the preceding aspects. Further, the composition comprises the cell according to any of the preceding aspects and a pharmaceutically acceptable carrier. The pharmaceutically acceptable carrier includes one or more of the following: pharmaceutically acceptable vehicle, disperser, additive, plasticizer, and excipient. Further, the composition may also comprise other therapeutic agents. In some embodiments, other therapeutic agents include, but are not limited to, chemotherapeutic agents, immunotherapeutic agents, or hormone therapeutic agents.

In a seventh aspect, the present invention provides a method of treating disease in a subject in need thereof, comprising administering to the subject an effective amount of the composition, or the CAR, or dual CAR, or the nucleic acid, or the vector, or the cell according to any of the preceding aspects.

In a further embodiment, the disease is ROR1 positive cancer; the disease is CD19 positive cancer; or both ROR1 and CD19 positive cancer. Preferably, the cancer is selected from one or more of blood cancer and solid cancer; preferably, the cancer includes, but is not limited to, gastric cancer, pancreatic cancer, esophageal cancer, lung cancer, ovarian cancer, head and neck cancer, bladder cancer, cervical cancer, sarcoma, cytoma, colon cancer, kidney cancer, colorectal cancer, liver cancer, melanoma, breast cancer, myeloma, neuroglioma, skin cancer, adrenal cancer, uterine cancer, testicular cancer, prostate cancer, blood cancer, leukemia, or lymphoma.

In an eighth aspect, the present invention provides a method of treating both ROR1 and CD19 positive cancer, comprising administering to the subject the dual CAR according to any of the preceding aspects; preferably, the cancer is selected from one or more of blood cancer and solid cancer; preferably, the cancer includes, but is not limited to, gastric cancer, pancreatic cancer, esophageal cancer, lung cancer, ovarian cancer, head and neck cancer, bladder cancer, cervical cancer, sarcoma, cytoma, colon cancer, kidney cancer, colorectal cancer, liver cancer, melanoma, breast cancer, myeloma, neuroglioma, skin cancer, adrenal cancer, uterine cancer, testicular cancer, prostate cancer, blood cancer, leukemia, or lymphoma.

In a ninth aspect, the present invention provides a method of producing a CAR-T cell comprising:
(1) introducing to a host cell the nucleic acid or the vector according to any of the preceding aspects, and
(2) isolating and/or expanding the CAR-T cells following the introduction.

The present application has the following advantages:
1) Jurkat NFAT-luciferase reporter cells expressing ROR1 CAR of the present application can be stimulated by ROR1-positive SK-Hep-1 Cells;
2) The ROR1 CAR of the present application has varied cytotoxicity against MCF7, HepG2, SK-Hep-1, and MDA-MB-231 target cells.
3) CD19-positive Raji Cells can stimulate the Jurkat NFAT-luciferase reporter cells expressing CD19 CAR of the present application;
4) The CD19 CARs of the present application have cytotoxicity against CD19-positive Raji, Jeko-1 and Nalm6 Cells;
5) Dual-targeting ROR1/CD19 CAR constructs may have improved therapeutic impact against double positive tumors with lower levels of cytokine release.

### DESCRIPTION OF DRAWINGS

FIG. 1 shows a structure of a chimeric antigen receptor (CAR). The CAR comprises from N-terminal to C-terminal: a CD8α signal peptide, a scFv antigen recognition domain (which was designed using the heavy chain variable region, followed by a flexible glycine/serine linker motif, and then a light chain variable region), a CD8α hinge domain, a CD8α transmembrane domain, a 4-1BB intracellular domain and a CD3ζ intracellular domain.

FIG. 2 shows an EGFP co-expression CAR PiggyBac Vector.

FIG. 3 shows a non-EGFP CAR PiggyBac Vector.

FIG. 4 shows expression of murine scFv CAR candidates in Jurkat NFAT luciferase cells. Jurkat reporter cells were electroporated with PiggyBac plasmids with the CAR cassette as well as PiggyBac Transposon mRNA. Integration was tracked over time. Shown here is CAR expression at day 1 post-electroporation. We initially screened six CARs with different anti-ROR1 scFv domains.

FIG. 5 shows Jurkat NFAT luciferase reporter activity measured in response to ROR1-positive SK-Hep-1 cells. Jurkat reporter cells expressing CAR constructs were cultured 1:1 overnight with SK-Hep-1 tumor cells. Luciferase activity was determined using NeoLite luciferase substrate and read out using a SpectraMax plate reader. CARs are represented by their antibody clone ID: m47, m829, m866, m38, m709, m508.

FIG. 6 shows Jurkat NFAT luciferase reporter activity measured in response to ROR1-negative MCF7 cells. Jurkat reporter cells expressing CAR constructs were cultured 1:1 overnight with MCF7 tumor cells. Luciferase activity was determined using NeoLite luciferase substrate and read out using a SpectraMax plate reader. This assay was performed to determine baseline CAR activity and off-target activation. CARs are represented by their antibody clone ID: m47, m829, m866, m38, m709, m508.

FIG. 7 shows Jurkat NFAT luciferase reporter activity measured in resting (unstimulated) cells to determine baseline NFAT activation. For many CAR constructs, receptor clustering and aggregation can cause T cell activation in the absence of stimuli. We therefore screened our CAR T cells for baseline luciferase activity. CARs are represented by their antibody clone ID: m47, m829, m866, m38, m709, m508.

FIG. 8 shows CAR expression and integration. Primary T cells derived from a healthy donor (ND22) were transduced with murine scFv CAR constructs. CAR expression and integration were tracked longitudinally. Shown here is the day 6 post-electroporation expression profile. CAR expression was determined using anti-mouse F(ab′)₂ antibody.

FIG. 9 shows CAR expression and integration. Primary T cells derived from a healthy donor (ND23) were transduced with murine scFv CAR constructs. CAR expression and integration were tracked longitudinally. Shown here is the day 11 post-electroporation expression profile. CAR expression was determined using anti-mouse F(ab′)₂ antibody.

FIG. 10 shows ROR1 expression profile. Dark gray histograms show the unlabeled control cells, anti-ROR1 labeled cells are shown as light gray histograms.

FIGS. 11A-11B show cytotoxicity of different CAR-T cells from different donors against different cell lines. CARs are represented by their antibody clone ID: m47, m829, m866, m38, m709, m508. FIG. 11A shows the results of CAR-T cells from donor ND22. FIG. 11B shows the results of CAR-T cells from donor ND23.

FIG. 12 shows expression of humanized ROR1 CAR constructs in Jurkat NFAT reporter cells. We designed three humanized CAR variants RC005a, RC005b, or RC005c based on the 709 humanized scFv. CAR expression was detected using anti-human F(ab′)₂.

FIG. 13 shows Jurkat NFAT luciferase reporter activity measured in response to ROR1-negative MCF7 cells. Jurkat reporter cells expressing CAR constructs were cultured 1:1 overnight with MCF7 tumor cells. Luciferase activity was determined using NeoLite luciferase substrate and read out using a SpectraMax plate reader. This assay was performed to determine baseline CAR activity and off-target activation.

FIG. 14 shows Jurkat NFAT luciferase reporter activity measured in response to ROR1-positive SK-Hep-1 cells. Jurkat reporter cells expressing CAR constructs were cultured 1:1 overnight with SK-Hep-1 tumor cells. Luciferase activity was determined using NeoLite luciferase substrate and read out using a SpectraMax plate reader.

FIG. 15 shows cytotoxicity of different CAR-T cells from primary T cells derived from a healthy donor (ND22) against different cell lines. CARs are represented by their antibody clone ID: R12, m709, hu709(VH2VL2), hu709(VH4VL1), hu709(VH4VL2).

FIG. 16 shows cytotoxicity of different CAR-T cells from primary T cells derived from a healthy donor (ND19) against different cell lines. CARs are represented by their antibody clone ID: R12, m709, hu709(VH2VL2), hu709(VH4VL1), hu709(VH4VL2).

FIG. 17 shows amount of IFN-γ released by the CAR-T cells from donor ND19.

FIG. 18A shows positive rate of T lymphocytes expressing different chimeric antigen receptors. FIGS. 18B-C show the experimental results of the specific killing of ROR1 CAR-T cells with different scFv on ROR1 positive tumor cells and ROR1 negative tumor cell line. FIGS. 18D-E show the release results of IFN-gamma cytokine in the supernatant of ROR1 CAR-T co-cultured with ROR1 positive and negative tumor cells of different scFvs.

FIG. 19A-19C shows humanized CD19 CAR expression. Jurkat NFAT-luciferase reporter cells were transduced by electroporation and CAR expression was tracked longitudinally. Shown here is CAR expression on day 3 post-electroporation.

FIG. 20 shows capacity for each humanized CD19 CAR variant to activate T cells. Jurkat NFAT luciferase reporter cells expressing the humanized FMC63 variants were cocultured overnight with CD19-expressing Raji cells. Luciferase activity was determined by the addition of NeoLite luciferase substrate and bioluminescence was read out using a SpectraMax plate reader.

FIG. 21 shows off-target CAR activity. Jurkat NFAT luciferase reporter cells expressing the humanized FMC63 variants were cocultured overnight with CD19-negative K562 cells. Luciferase activity was determined by the addition of NeoLite luciferase substrate and bioluminescence was read out using a SpectraMax plate reader. LSL008d and LS008f showed high background activity.

FIG. 22 shows the cytotoxicity of CD19 CAR constructs against the CD19-positive cell line Raji.

FIG. 23 shows the cytotoxicity of CD19 CAR constructs against the CD19-positive cell line Jeko-1.

FIG. 24 shows the cytotoxicity of CD19 CAR constructs against the CD19-positive cell line Nalm6.

FIG. 25 shows the cytotoxicity of CD19 CAR constructs against the CD19-negative cell line K562.

FIG. 26 shows structural schematic of the Dual CAR Cassettes.

FIG. 27 A-27B shows CAR expression in Jurkat NFAT luciferase reporter cells.

FIG. 28 shows very low NFAT reporter activity in response to the ROR1 and CD19 double negative cell line K562.

FIG. 29 shows a high level of NFAT reporter activity in RC025 and RC026 relative to the LS008 control cells in response to CD19-positive cell line Raji cells

FIG. 30 shows strong NFAT activity in all the CAR constructs tested in response to ROR1/CD19 double positive cell line Jeko-1.

FIG. 31A-31B shows CAR expression in primary T cells.

FIG. 32 shows cytotoxicity of CAR constructs against ROR1/CD19 double negative cell line MCF7.

FIG. 33 shows cytotoxicity of CAR constructs against ROR1-positive cell line MDA-MB-231.

FIG. 34 shows cytotoxicity of CAR constructs against ROR1/CD19 double positive cell line Jeko-1.

FIG. 35 shows ROR1 and CD19 expression of primary tumor cells derived from a DLBCL patient.

FIG. 36 shows cytotoxicity of CAR-T cells against patient-derived DLBCL tumor cells

FIG. 37 shows amount of IFN-γ released by the CAR T cells.

### DETAILED DESCRIPTION

### Definitions

For purposes of interpreting the CAR or dual CAR used in the following examples, the following definitions are provided.

### 1. Definition of Cars Used in the Following Examples

1.1 CAR with CD8 Transmembrane Domain

### CD8α SP-VH-(GGGGS)₃ Linker-VL-CD8α Hinge Domain-CD8α Transmembrane Domain-4-1BB Intracellular Domain-CD3ζ Intracellular Domain

### 1) Murine ROR1-CARs:

m38 CAR: CD8α SP-m38VH-(GGGGS)₃ linker-m38VL-CD8α hinge domain-CDSatransmembrane domain-4-1BB intracellular domain-CD3ζ intracellular domain
m47 CAR: CD8α SP-m47VH-(GGGGS)₃ linker-m47VL-CD8α hinge domain-CD8α transmembrane domain-4-1BB intracellular domain-CD3ζ intracellular domain
m508 CAR: CD8α SP-m508VH-(GGGGS)₃ linker-m508VL-CD8α hinge domain-CD8α transmembrane domain-4-1B13 intracellular domain-CD3ζ intracellular domain
m709 CAR (i.e. RC005): CD8α SP-m709VH-(GGGGS)₃linker-m709VL-CD8α hinge domain-CD8α transmembrane domain-4-1BB intracellular domain-CD3ζ intracellular domain
m829 CAR: CD8α SP-m829VH-(GGGGS)₃ linker-m829VL-CD8α hinge domain-CD8α transmembrane domain-4-1BB intracellular domain-CD3ζ intracellular domain
m866 CAR: CD8α SP-m866VH-(GGGGS)₃ linker-m866VL-CD8α hinge domain-CD8α transmembrane domain-4-1BB intracellular domain-CD3ζ intracellular domain
   The VH and VL sequences of six mouse anti-ROR1 monoclonal antibodies m38, m47, m508, m709, m829 and m866 are shown in Table 1:

TABLE 1

| | | SEQ |
|---|---|---|
| DESCRIP- | | ID |
| TION | SEQUENCE | NO. |
| anti- | QVQLQQSGAELVKPGASVKLSCTASGFNIKD | 44 |
| ROR1 | TYMHWVKQRPEQGLEWIGRIDPANGNTKYDP | |
| clone | KFQGKATITADTSSNTAYLQLSSLTSEDTAV | |
| m38-VH | YYCARTEGAMDYWGQGTSVTVSA | |
| anti- | EVKLMESGGGLVKPGGSLKLSCAASGFTFSD | 45 |
| ROR1 | YAMSWVRQTPERRLEWVASISTGASTYYPDS | |
| clone | VKGRFTISRDNARNILYLQMSSLRSEDTAMY | |
| m47-VH | YCANYDPSYWYFDVWGAGTTVTVSS | |
| anti- | QVQLQQSGAELVRSGASVKLSCTASGFNIKD | 46 |
| ROR1 | YYMHWVKQRPEQGLEWIGYIDPEIGDTEYAP | |
| clone | KFQGKATMTADTSSNTAYLQLSSLTSEDTAV | |
| m508-VH | YYCRVDPLYDGYYDYWGQGTTLTVSS | |
| anti- | DVQLQESGAELVRPGASVTLSCKASGYTFTD | 47 |
| ROR1 | YEMHWVKQTPVHGLEWIGAIDPETGGTAYNQ | |
| clone | KFKGKATLTADKSSSTAYMELRSLTSEDSAV | |
| m709-VH | YYCTPYYGYAMDYWGQGTSVTVSS | |
| anti- | EVQLKESGPGLVKPSQSLSLTCTVTGYSITS | 48 |
| ROR1 | DYAWNWIRQFPGNKLEWMGYISYSGSTSYNP | |
| clone | SLKSRISITRDTSKNQFFLQLNSVTTEDTAT | |
| m829-VH | YYCARRDYDVAMDYWGQGTSVTVSS | |
| anti- | QVQLQQSGAELVRPGASVKLSCKALGYTFTD | 49 |
| ROR1 | YEMHWVKQTPVHGLEWIGGIHQGSGGTAYNQ | |
| clone | KFKGKATLTADKSSSTAYMELSSLTSEDSAV | |
| m866-VH | YYCTRDYYDYDGFAYWGQGTLVTVSS | |
| anti- | DIVLTQSPATLSVTPGDRVSLSCRASQSISD | 50 |
| ROR1 | YLHWYQQKSHESPRLLIKYASQSISGIPSRF | |
| clone | SGSGSGTDFTLSINSVEPEDVGVYYCQNGHS | |
| m38-VL | FPLTFGAGTKLEIK | |
| anti- | DIQMTQSPSSMYASLGERVTITCKASQDINS | 51 |
| ROR1 | YLSWFQQKPGKSPKTLIYRANRLVDGVPSRF | |
| clone | SGSGSGQDYSLTISSLEYEDMGIYYCLQYDE | |
| m47-VL | FPYTFGGGTKLDMK | |
| anti- | DIVMTQSHKFMSTSVGDRVSITCKASQDVST | 52 |
| ROR1 | AVAWYQQKPGQSPKLLIYSASYRYTGVPDRF | |
| clone | TGSGSGTDFTFTISSVQAEDLAVYYCQQHYS | |
| m508-VL | TPPTFGAGTKLDLK | |
| anti- | DIVMTQSQKFMSTSVGDRVSVTCKASQNVGT | 53 |
| ROR1 | NVAWYQQKPGQSPKLLIYWASTRHTGVPDRF | |
| clone | TGSGSGTDFTLTISNVQSEDLADYFCQQYSS | |
| m709-VL | YPLTFGAGTKLEIK | |
| anti- | DIELTQSPASLAVSLGQRATISCKASQSVDY | 54 |
| ROR1 | DGDSYMNWYQQKPGQPPKLLIYAASNLESGI | |
| clone | PARFSGSGSGTDFTLNIHPVEEEDAATYYCQ | |
| m829-VL | QGNEDPYTFGGGTKLEIK | |
| anti- | DIVLSQSPAILSASPGEKVTMTCRTSSSVSY | 55 |
| ROR1 | MHWYQQKPGSSPKPWIYATSNLASGVPARFS | |
| clone | GSGSGTSYSLTISRVEAEDAATYYCQQWSSN | |
| m866-VL | PPTFGGGTKLEIK | |

The sequences of 6 CDR regions of VH and VL for six mouse anti-ROR1 monoclonal antibodies m38, m47, m508, m709, m829 and m866 are shown in Table 2, the analysis system is IMGT system.

TABLE 2

| | | SEQ |
|---|---|---|
| | | ID |
| DESCRIPTION | SEQUENCE | NO. |
| anti-ROR1 clone m38-HCDR1 | GFNIKDTY | 1 |
| anti-ROR1 clone m38-HCDR2 | IDPANGNT | 2 |
| anti-ROR1 clone m38-HCDR3 | ARTEGAMDY | 3 |
| anti-ROR1 clone m47-HCDR1 | GFTFSDYA | 4 |
| anti-ROR1 clone m47-HCDR2 | ISTGAST | 5 |
| anti-ROR1 clone m47-HCDR3 | ANYDPSYWYFDV | 6 |
| anti-ROR1 clone m508-HCDR1 | GFNIKDYY | 7 |
| anti-ROR1 clone m508-HCDR2 | IDPEIGDT | 8 |
| anti-ROR1 clone m508-HCDR3 | RVDPLYDGYYDY | 9 |
| anti-ROR1 clone m709-HCDR1 | GYTFTDYE | 10 |
| anti-ROR1 clone m709-HCDR2 | IDPETGGT | 11 |
| anti-ROR1 clone m709-HCDR3 | TPYYGYAMDY | 12 |
| anti-ROR1 clone m829-HCDR1 | GYSITSDYA | 13 |
| anti-ROR1 clone m829-HCDR2 | ISYSGST | 14 |
| anti-ROR1 clone m829-HCDR3 | ARRDYDVAMDY | 15 |
| anti-ROR1 clone m866-HCDR1 | GYTFTDYE | 10 |
| anti-ROR1 clone m866-HCDR2 | IHQGSGGT | 16 |
| anti-ROR1 clone m866-HCDR3 | TRDYYDYDGFAY | 17 |
| anti-ROR1 clone m38-LCDR1 | QSISDY | 18 |
| anti-ROR1 clone m38-LCDR2 | YAS | 19 |
| anti-ROR1 clone m38-LCDR3 | QNGHSFPLT | 20 |
| anti-ROR1 clone m47-LCDR1 | QDINSY | 21 |
| anti-ROR1 clone m47-LCDR2 | RAN | 22 |
| anti-ROR1 clone m47-LCDR3 | LQYDEFPYTFGGGTK | 23 |
| anti-ROR1 clone m508-LCDR1 | QDVSTA | 24 |
| anti-ROR1 clone m508-LCDR2 | SAS | 25 |
| anti-ROR1 clone m508-LCDR3 | QQHYSTPPTFGAGTK | 26 |
| anti-ROR1 clone m709-LCDR1 | QNVGTN | 27 |
| anti-ROR1 clone m709-LCDR2 | WAS | 28 |
| anti-ROR1 clone m709-LCDR3 | QQYSSYPLT | 29 |
| anti-ROR1 clone m829-LCDR1 | QSVDYDGDSY | 30 |
| anti-ROR1 clone m829-LCDR2 | AAS | 31 |
| anti-ROR1 clone m829-LCDR3 | QQGNEDPYT | 32 |
| anti-ROR1 clone m866-LCDR1 | SSVSY | 33 |
| anti-ROR1 clone m866-LCDR2 | ATS | 34 |
| anti-ROR1 clone m866-LCDR3 | QQWSSNPPT | 35 |

### 2) Humanized ROR1-CARs

RC005a: CD8α SP-hu709 VH2VL2-CD8α hinge domain-CD8α transmembrane domain-4-11BB intracellular domain-CD3ζ intracellular domain
RC005b: CD8α SP-hu709 VH4VL1-CD8α hinge domain-CD8a transmembrane domain-4-11BB intracellular domain-CD3ζ intracellular domain
RC005c: CD8α SF-hu709 VH4VL2-CD8α hinge domain-CD8α transmembrane domain-4-11BB intracellular domain-CD3ζ intracellular domain
1720: CD8α SP-1720 VH VL-CD8α hinge domain-CD8α transmembrane domain-4-1BB intracellular domain-CD3ζ intracellular domain

The sequences of VH and VL of Humanized anti-ROR1 hu709 and clone 1720 are shown in Table 3 (Underlined Sequences represent CDRs, the analysis system is IMGT system).

TABLE 3

| | | SEQ |
|---|---|---|
| DESCRIP- | | ID |
| TION | SEQUENCE | NO. |
| humanized | EVQLVQSGAEVKKPGATVKISCKVSGYTFTDYE | 56 |
| anti-ROR1 | MHWVQQAPGKGLEWIGAIDPETGGTAYNQKFKG | |
| clone | RATITADTSTDTAYMELSSLRSEDTAVYYCTPY | |
| hu709 VH2 | YGYAMDYWGQGTLVTVSS | |
| humanized | EVQLVQSGAEVKKPGATVKISCKVSGYTFTDYE | 57 |
| anti-ROR1 | MHWVKQAPGKGLEWIGAIDPETGGTAYNQKFKG | |
| clone | RATITADKSSSTAYMELSSLRSEDSAVYYCTPY | |
| hu709 VH4 | YGYAMDYWGQGTLVTVSS | |
| humanized | DIQLTQSPSFLSASVGDRVTITCKASQNVGTNVA | 58 |
| anti-ROR1 | WYQQKPGKAPKLLIYWASTRHTGVPSRFSGSGS | |
| clone | GTEFTLTISSLQPEDFATYYCQQYSSYPLTFGQG | |
| hu709 VL1 | TKLEIK | |
| humanized | DIQLTQSPSFLSASVGDRVTITCKASQNVGTNVA | 59 |
| anti-ROR1 | WYQQKPGKAPKLLIYWASTRHTGVPSRFSGSGS | |
| clone | GTEFTLTISSLQPEDFATYFCQQYSSYPLTFGQG | |
| hu709 VL2 | TKLEIK | |
| humanized | EVQLVQSGGGVVQPGGSLRLSCAASGFTFSSYT | 81 |
| anti-ROR1 | MHWVRQAPGKGLEWVAVISFDGSSKYYADSV | |
| clone | KGRFTISRDNSKNTLYLQMNSLRAEDTAVYYCA | |
| 1720-VH | SDQAWGYFDYWGQGTLVTVSS | |
| humanized | EIVLTQSPGTLSLSPGERATLSCRASQSVSSSY | 82 |
| anti-ROR1 | LAWYQQKPGQAPRLLIYGASSRATGIPDRFSGSG | |
| clone | SGTDFTLTISRLEPEDFAVYYCQQYGSSPGTFGQ | |
| 1720-VL | GTKVDIK | |

The sequences of CDRs of VH and VL of hu709 and clone 1720 are shown in Table 4.

TABLE 4

| | | SEQ |
|---|---|---|
| | | ID |
| DESCRIPTION | SEQUENCE | NO. |
| humanized anti-ROR1 clone | GYTFTDYE | 10 |
| hu709 HCDR1 | | |
| humanized anti-ROR1 clone | IDPETGGT | 11 |
| hu709 HCDR2 | | |
| humanized anti-ROR1 clone | ATYYGYAMDY | 36 |
| hu709 HCDR3 | | |
| humanized anti-ROR1 clone | TPYYGYAMDY | 12 |
| hu709 HCDR3′ | | |
| humanized anti-ROR1 clone | QNVGTN | 27 |
| hu709 LCDR1 | | |
| humanized anti-ROR1 clone | WAS | 28 |
| hu709 LCDR2 | | |
| humanized anti-ROR1 clone | QQYSSYPLT | 29 |
| hu709 LCDR3 | | |
| anti-ROR1 clone 1720-HCDR1 | GFTFSSYT | 83 |
| anti-ROR1 clone 1720-HCDR2 | ISFDGSSK | 84 |
| anti-ROR1 clone 1720-HCDR3 | ASDQAWGYFDY | 85 |
| anti-ROR1 clone 1720-LCDR1 | QSVSSSY | 86 |
| anti-ROR1 clone 1720-LCDR2 | GAS | 87 |
| anti-ROR1 clone 1720-LCDR3 | QQYGSSPGT | 88 |

### 3) Benchmark ROR1-CAR: R12 ROR1 CAR

R12: CD8α SP-R12 VH VL-CD8α hinge domain-CD8α transmembrane domain-4-1BB intracellular domain-CD3ζ intracellular domain

The sequences of VH and VL of R12 are shown in Table 5.

TABLE 5

| | | SEQ |
|---|---|---|
| DESCRIP- | | ID |
| TION | SEQUENCE | NO. |
| R12 VH | QEQKVESGGRLVTPGGSLTLSCKASGFDFSAYY | 79 |
| | MSWVRQAPGKGLEWIATIYPSSGKTYYATWVN | |
| | GRFTISSDNAQNTVDLQMNSLTAADRATYFCAR | |
| | DSYADDGALFNIWGPGTLVTISS | |
| R12 VL | ELVLTQSPSVSAALGSPAKITCTLSSAHKTDTI | 80 |
| | DWYQQLQGEAPRYLMQVQSDGSYTKRPGVPDRF | |
| | SGSSSGADRYLIIPSVQADDEADYYCGADYIGG | |
| | YVFGGGTQLTVTG | |

### 4) One Murine CD19 CAR Variant

LS008: CD8α SP-FMC63 VH VL-CD8α hinge domain-CD8α transmembrane domain-4-1BB intracellular domain-CD3ζ intracellular domain

The sequences of VH and VL of FMC63 are shown in Table 6 (Underlined Sequences represent CDRs, the analysis system is IMGT system).

TABLE 6

| | | SEQ |
|---|---|---|
| DESCRIP- | | ID |
| TION | SEQUENCE | NO. |
| FMC63 | EVKLQESGPGLVAPSQSLSVTCTVSGVSLPDYG | 69 |
| (CD19) | VSWIRQPPRKGLEWLGVIWGSETTYYNSALKSR | |
| VH | LTIIKDNSKSQVFLKMNSLQTDDTAIYYCAKHY | |
| | YYGGSYAMDYWGQGTSVTVSS | |
| FMC63 | DIQMTQTTSSLSASLGDRVTISCRASQDISKYLN | 70 |
| (CD19) | WYQQKPDGTVKLLIYHTSRLHSGVPSRFSGSGS | |
| VL | GTDYSLTISNLEQEDIATYFCQQGNTLPYTFGGG | |
| | TKLEIT | |

The sequences of CDRs of VH and VL of FMC63 are shown in Table 7.

TABLE 7

| | | SEQ |
|---|---|---|
| | | ID |
| DESCRIPTION | SEQUENCE | NO. |
| FMC63 (CD19) VH-HCDR1 | GVSLPDYG | 37 |
| FMC63 (CD19) VH-HCDR2 | IWGSETT | 38 |
| FMC63 (CD19) VH-HCDR3 | AKHYYYGGSYAMDY | 39 |
| FMC63 (CD19) VL-LCDR1 | QDISKY | 41 |
| FMC63 (CD19) VL-LCDR2 | HTS | 42 |
| FMC63 (CD19) VL-LCDR3 | QQGNTLPYT | 43 |

### 5) 16 Humanized CD19 CAR Variants

LS008a: CD8α SP-Humanized FMC63 VII version 1-VL version 1-CD8α hinge domain-CD8α transmembrane domain-4-1BB intracellular domain-CD3ζ intracellular domain
LS008b: CD8α SP-Humanized FMC63 VH version 1-VL version 2-CD8α hinge domain-CD8α transmembrane domain-4-1BB intracellular domain-CD3ζ intracellular domain
LS008c: CD8α SP-Humanized FMC63 VH version 1-VL version 3-CD8α hinge domain-CD8α transmembrane domain-4-1BB intracellular domain-CD3ζ intracellular domain
LS008d: CD8α SP-Humanized FMC63 VH version 1-VL version 4-CD8α hinge domain-CD8α transmembrane domain-4-1BB intracellular domain-CD3ζ intracellular domain
LS008e: CD8α SP-Humanized FMC63 VH version 2-VL version 1-CD8α hinge domain-CD8α transmembrane domain-4-1BB intracellular domain-CD3ζ intracellular domain
LS008f: CD8α SP-Humanized FMC63 VH version 2-VL version 2-CD8α hinge domain-CD8α transmembrane domain-4-1BB intracellular domain-CD3ζ intracellular domain
LS008g: CD8α SP-Humanized FMC63 VH version 2-VL version 3-CD8α hinge domain-CD8α transmembrane domain-4-1BB intracellular domain-CD3ζ intracellular domain
LS008h: CD8α SP-Humanized FMC63 VH version 2-VL version 4-CD8α hinge domain-CD8α transmembrane domain-4-1BB intracellular domain-CD3ζ intracellular domain
LS008i: CD8α SP-Humanized FMC63 VH version 3-VL version 1-CD8α hinge domain-CD8α transmembrane domain-4-1BB intracellular domain-CD3ζ intracellular domain
LS008j: CD8α SP-Humanized FMC63 VH version 3-VL version 2-CD8α hinge domain-CD8α transmembrane domain-4-1BB intracellular domain-CD3ζ intracellular domain
LS008k: CD8α SP-Humanized FMC63 VH version 3-VL version 3-CD8α hinge domain-CD8α transmembrane domain-4-1BB intracellular domain-CD3ζ intracellular domain
LS008i: CD8α SP-Humanized FMC63 VH version 3-VL version 4-CD8α hinge domain-CD8α transmembrane domain-4-1BB intracellular domain-CD3ζ intracellular domain
LS008m: CD8α SP-Humanized FMC63 VH version 4-VL version 1-CD8α hinge domain-CD8α transmembrane domain-4-1BB intracellular domain-CD3ζ intracellular domain
LS008n: CD8α SP-Humanized FMC63 VH version 4-VL version 2-CD80C hinge domain-CD8α transmembrane domain-4-1BB intracellular domain-CD3ζ intracellular domain
LS008o: CD8α SP-Humanized FMC63 VH version 4-VL version 3-CD8α hinge domain-CD8α transmembrane domain-4-1BB intracellular domain-CD3ζ intracellular domain
LS008p: CD8α SP-Humanized FMC63 VH version 4-VL version 4-CD8α hinge domain-CD8α transmembrane domain-4-1BB intracellular domain-CD3ζ intracellular domain

The sequences of VH and VL of FMC63 are shown in Table 8.

TABLE 8

| | | SEQ |
|---|---|---|
| DESCRIPT- | | ID |
| ION | SEQUENCE | NO. |
| Humanized | QVQLQESGPGLVKPSETLSLTCTVSGVSLPDYG | 71 |
| FMC63 | VSWIRQPPGKGLEWIGVIWGSETTYYNSALKSR | |
| (CD19) VH | VTISVDTSKNQFSLKLSSVTAADTAVYYCARHY | |
| version 1 | YYGGSYAMDYWGQGTLVTVSS | |
| Humanized | QVQLQESGPGLVKPSETLSLTCTVSGVSLPDYG | 72 |
| FMC63 | VSWIRQPPGKGLEWLGVIWGSETTYYNSALKSR | |
| (CD19) VH | VTISKDTSKNQFSLKLSSVTAADTAVYYCAKHY | |
| version 2 | YYGGSYAMDYWGQGTLVTVSS | |
| Humanized | QVQLQESGPGLVKPSETLSLTCTVSGVSLPDYG | 73 |
| FMC63 | VSWIRQPPGKGLEWLGVIWGSETTYYNSALKSR | |
| (CD19) VH | LTISKDTSKSQFSLKLSSVTAADTAVYYCAKHY | |
| version 3 | YYGGSYAMDYWGQGTLVTVSS | |
| Humanized | QVQLQESGPGLVKPSETLSLTCTVSGVSLPDYG | 74 |
| FMC63 | VSWIRQPPGKGLEWLGVIWGSETTYYNSALKSR | |
| (CD19) VH | LTISKDTSKSQVFLKLSSVTAADTAVYYCAKHY | |
| version 4 | YYGGSYAMDYWGQGTLVTVSS | |
| Humanized | DIQMTQSPSSLSASVGDRVTITCRASQDISKYLN | 75 |
| FMC63 | WYQQKPGKAPKLLIYHTSRLHSGVPSRFSGSGS | |
| (CD19) VL | GTDFTFTISSLQPEDIATYYCQQGNTLPYTFGGG | |
| version 1 | TKLEIK | |
| Humanized | DIQMTQSPSSLSASVGDRVTITCRASQDISKYLN | 76 |
| FMC63 | WYQQKPGKAPKLLIYHTSRLHSGVPSRFSGSGS | |
| (CD19) VL | GTDFTFTISSLQPEDIATYFCQQGNTLPYTFGGG | |
| version 2 | TKLEIK | |
| Humanized | DIQMTQSPSSLSASVGDRVTITCRASQDISKYLN | 77 |
| FMC63 | WYQQKPGKAVKLLIYHTSRLHSGVPSRFSGSGS | |
| (CD19) VL | GTDYTFTISSLQPEDIATYFCQQGNTLPYTFGGG | |
| version 3 | TKLEIK | |
| Humanized | DIQMTQSPSSLSASVGDRVTITCRASQDISKYLN | 78 |
| FMC63 | WYQQKPDKAVKLLIYHTSRLHSGVPSRFSGSGS | |
| (CD19) VL | GTDYTFTISSLQPEDIATYFCQQGNTLPYTFGGG | |
| version 4 | TKLEIK | |

The sequences of CDRs of VH and VL of FMC63 are shown in Table 9.

TABLE 9

| | | SEQ |
|---|---|---|
| | | ID |
| DESCRIPTION | SEQUENCE | NO. |
| Humanized FMC63 | GVSLPDYG | 37 |
| (CD19) VH | | |
| version 1-HCDR1 | | |
| Humanized FMC63 | IWGSETT | 38 |
| (CD19) VH | | |
| version 1-HCDR2 | | |
| Humanized FMC63 | ARHYYYGGSYAMDY | 40 |
| (CD19) VH | | |
| version 1-HCDR3 | | |
| Humanized FMC63 | GVSLPDYG | 37 |
| (CD19) VH | | |
| version 2-HCDR1 | | |
| Humanized FMC63 | IWGSETT | 38 |
| (CD19) VH | | |
| version 2-HCDR2 | | |
| Humanized FMC63 | AKHYYYGGSYAMDY | 39 |
| (CD19) VH | | |
| version 2-HCDR3 | | |
| Humanized FMC63 | GVSLPDYG | 37 |
| (CD19) VH | | |
| version 3-HCDR1 | | |
| Humanized FMC63 | IWGSETT | 38 |
| (CD19) VH | | |
| version 3-HCDR2 | | |
| Humanized FMC63 | AKHYYYGGSYAMDY | 39 |
| (CD19) VH | | |
| version 3-HCDR3 | | |
| Humanized FMC63 | GVSLPDYG | 37 |
| (CD19) VH | | |
| version 4-HCDR1 | | |
| Humanized FMC63 | IWGSETT | 38 |
| (CD19) VH | | |
| version 4-HCDR2 | | |
| Humanized FMC63 | AKHYYYGGSYAMDY | 39 |
| (CD19) VH | | |
| version 4-HCDR3 | | |
| Humanized FMC63 | QDISKY | 41 |
| (CD19) VL | | |
| version 1-LCDR1 | | |
| Humanized FMC63 | HTS | 42 |
| (CD19) VL | | |
| version 1-LCDR2 | | |
| Humanized FMC63 | QQGNTLPYT | 43 |
| (CD19) VL | | |
| version 1-LCDR3 | | |
| Humanized FMC63 | QDISKY | 41 |
| (CD19) VL | | |
| version 2-LCDR1 | | |
| Humanized FMC63 | HTS | 42 |
| (CD19) VL | | |
| version 2-LCDR2 | | |
| Humanized FMC63 | QQGNTLPYT | 43 |
| (CD19) VL | | |
| version 2-LCDR3 | | |
| Humanized FMC63 | QDISKY | 41 |
| (CD19) VL | | |
| version 3-LCDR1 | | |
| Humanized FMC63 | HTS | 42 |
| (CD19) VL | | |
| version 3-LCDR2 | | |
| Humanized FMC63 | QQGNTLPYT | 43 |
| (CD19) VL | | |
| version 3-LCDR3 | | |
| Humanized FMC63 | QDISKY | 41 |
| (CD19) VL | | |
| version 4-LCDR1 | | |
| Humanized FMC63 | HTS | 42 |
| (CD19) VL | | |
| version 4-LCDR2 | | |
| Humanized FMC63 | QQGNTLPYT | 43 |
| (CD19) VL | | |
| version 4-LCDR3 | | |

2) CAR with Transmembrane (Tm) Linking Juxtamembrane (Jm) Domain Instead of CD8 Transmembrane Domain

### ROR1-CAR:

ROR1scFv (hu709 VH4VL2)-CD8Hinge-SEZ6L2 tm jm-CD35

### CD19-CAR:

CD19 scFv (FMC63)-CD8Hinge-SEZ6L2 tm jm-CD35

### 4. Definition of Dual CAR Used in the Following Examples

RC025 CAR comprises from N-Terminal to C-Terminal: CD19 scFv (FMC63)-CD8Hinge-CD8 tm-4-1BB-CD3ζ-P2A-ROR1scFv (Hu709 VH4VL2)-CD8Hinge-SEZ6L2 tm Jm-CD3ζ-GS linker-GFP;
RC026 CAR comprises from N-terminal to C-terminal: ROR1scFv (hu709 VH4VL2)-CD8Hinge-CD8 tm-4-1BB-CD3ζ-P2A-CD19 scFv (FMC63)-CD8Hinge-SEZ6L2 tm jm-CD3ζ-GS linker-GFP.

### Bench CAR for Dual CAR RC025 and RC026:

LS008 (Murine CD19 CAR): CD8a SP-FMC63 VH VL-CD8a hinge domain-CD8a transmembrane domain-4-1BB intracellular domain-CD3 ζ intracellular domain
RC005c (Humanized ROR1-CAR): CD8a SP-hu709 VH4VL2-CD8a hinge domain-CD8a transmembrane domain-4-1BB intracellular domain-CD3 (intracellular domain

### 5. Other Parts Used in the CAR

The sequences of other parts used in the CAR are shown in Table 10.

TABLE 10

| | | SEQ |
|---|---|---|
| DESCRIP- | | ID |
| TION | SEQUENCE | NO. |
| linker | GGGGSGGGGSGGGGS | 60 |
| CD8α | MALPVTALLLPLALLLHAARP | 61 |
| signal | | |
| peptide | | |
| CD8α | TTTPAPRPPTPAPTIASQPLSLRPEACRPAAGGAV | 62 |
| hinge | HTRGLDFACD | |
| domain | | |
| CD8α | IYIWAPLAGTCGVLLLSLVITLYC | 63 |
| trans- | | |
| membrane | | |
| region | | |
| domain | | |
| 4-1BB | KRGRKKLLYIFKQPFMRPVQTTQEEDGCSCRFP | 64 |
| intra- | EEEEGGCEL | |
| cellular | | |
| domain | | |
| (4-1BB) | | |
| CD3ζ | RVKFSRSADAPAYKQGQNQLYNELNLGRREEY | 65 |
| intra- | DVLDKRRGRDPEMGGKPRRKNPQEGLYNELQK | |
| cellular | DKMAEAYSEIGMKGERRRGKGHDGLYQGLSTA | |
| domain | TKDTYDALHMQALPPR | |
| (CD3ζ) | | |
| Seizure | LALAILLPLGLVIVLGSGVYIYYTKLQGKSLFGFS | 66 |
| 6-like | GSHSYSPITVESDFSNPLY | |
| protein | | |
| 2 | | |
| trans- | | |
| membrane | | |
| and | | |
| juxta- | | |
| membrane | | |
| domain | | |
| EGFP | MVSKGEELFTGVVPILVELDGDVNGHKFSVSGE | 67 |
| (GFP) | GEGDATYGKLTLKFICTTGKLPVPWPTLVTTLT | |
| | YGVQCFSRYPDHMKQHDFFKSAMPEGYVQERT | |
| | IFFKDDGNYKTRAEVKFEGDTLVNRIELKGIDFK | |
| | EDGNILGHKLEYNYNSHNVYIMADKQKNGIKV | |
| | NFKIRHNIEDGSVQLADHYQQNTPIGDGPVLLPD | |
| | NHYLSTQSALSKDPNEKRDHMVLLEFVTAAGIT | |
| | LGMDELYK | |
| P2A | GSGATNFSLLKQAGDVEENPGP | 68 |

### EXAMPLES

### Example 1: Design and Humanization of Human ROR1-Specific Chimeric Antigen Receptor

### 1. Design of ROR1 CAR Candidates

ROR1 CAR constructs were designed according to the following structure: CD8α SP-VH-(GGGGS)₃ linker-VL-CD8a hinge domain-CD8a transmembrane domain-4-1BB intracellular domain-CD3 intracellular domain (FIG. 1).

Preparation of CAR constructs is a common technical method in the art. For example, first, CAR gene fragments are prepared through gene synthesis technology, then CAR PiggyBac transposon expression vectors are constructed, CAR constructs are electroporated into a target cell, and construct expression is assessed by flow cytometry or total protein analysis.

2. ROR1 CAR Candidates with Murine ROR1-scFv (m38, m47, m508, m709, m829, and m866)

We designed six ROR1 CAR candidates based on the murine anti-human ROR1-specific monoclonal antibody clones (m38, m47, m508, m709, m829, and m866) and named them as m38 CAR, m47 CAR, m508 CAR, m709 CAR, m829 CAR and m866 CAR respectively. All six ROR1 CAR constructs were designed according to the above structure: CD8α SP-VH-(GGGGS)₃ linker-VL-CD8α hinge domain-CD8α transmembrane domain-4-1B313 intracellular domain-CD3ζ intracellular domain (FIG. 1).

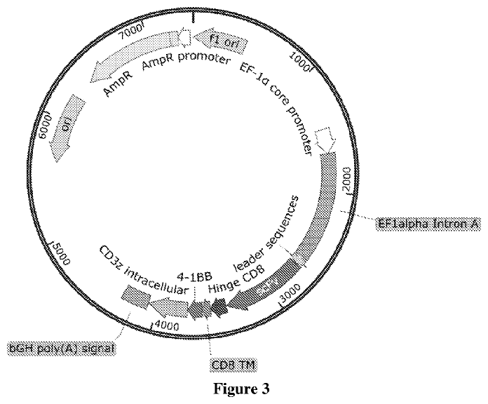

The sequence of each of the six ROR1 CAR candidates with murine ROR1-scFv were subcloned into the PiggyBac vector flanked by inverted terminal repeats (ITRs) to mediate construct integration into the host cell genome. CAR expression was driven by an EFI1a promoter upstream of the CAR sequence. CAR membrane trafficking was mediated by the CD8α signal peptide at the 5′ end of the CAR sequence. The plasmid structure is shown in FIG. 3 (non-EGFP plasmid, synthesized by Genscript).

2.1. Validation of ROR1 CAR Candidates with Murine ROR1-scFv (m38, m47, m508, m709, m829, and m866)

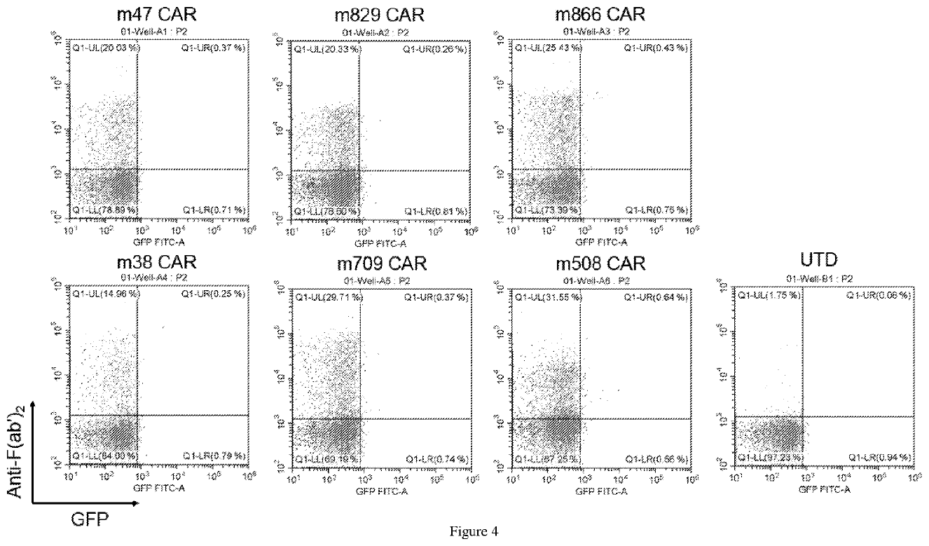

To screen for CAR activity, Jurkat NFAT-Luciferase reporter cells were electroporated with PiggyBac plasmids with the CAR cassette corresponding to each of the ROR1 CAR candidates as well as PiggyBac Transposon mRNA. CAR surface expression was determined using Alexa Fluor 647-conjugated goat anti-mouse IgG F(ab′)₂ antibodies (Jackson ImmunoResearch, 115-605-006) and visualized by flow cytometry using a BD Cytoflex flow cytometer (FIG. 4). In FIG. 4, the GFP^{Low}, F(ab′)₂^{Low} cells are the untransduced population (lower left). The GFP^{Low}, F(ab′)₂^{High} are the CAR positive cells (upper left). The UTD group are the untransduced Jurkat NFAT-Luciferase reporter cells. It can be seen from FIG. 4 that each of the six murine scFv CAR has been expressed on the surface of Jurkat NFAT-Luciferase reporter cells.

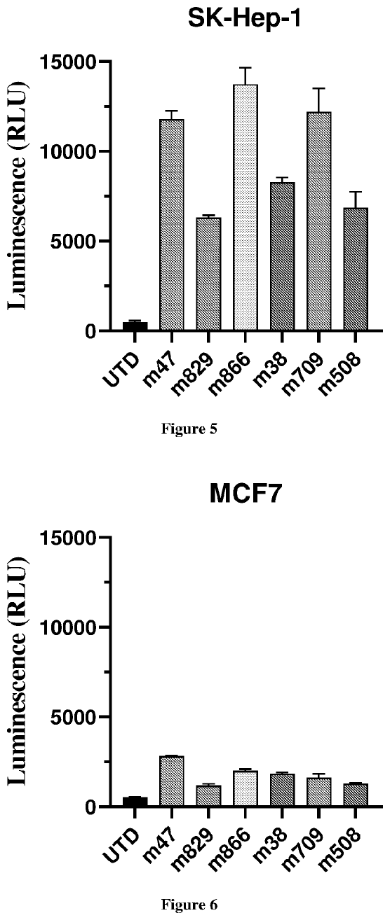

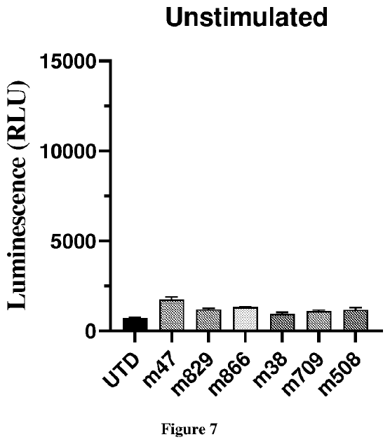

Once CAR expression was confirmed, we screened the ROR1 CAR candidates by NFAT-luciferase reporter assay to determine the lead ROR1 CAR candidates. Jurkat NFAT reporter cells expressing m38 CAR, m47 CAR, m508 CAR, m709 CAR, m829 CAR, or m866 CAR were mixed 1:1 with ROR1-positive SK-Hep-1 Cells (FIG. 5) or ROR1-negative MCF7 cells (FIG. 6). Jurkat T cells and target cells (ROR1-positive SK-Hep-1 Cells or ROR1-negative MCF7 cells) were co-cultured overnight and then luminescence was determined using NeoLite (Perkins Elmer) Luciferase substrate. It can be seen from FIGS. 5-6 that only ROR1-positive SK-Hep-1 Cells can stimulate the Jurkat NFAT-luciferase reporter cells, and Jurkat/NFAT activation indicates that efficient TCR signaling was initiated. Unstimulated Jurkat NFAT-luciferase reporter cells (UTD) expressing the ROR1 CAR constructs were used to determine CAR baseline signaling activity (FIG. 7).

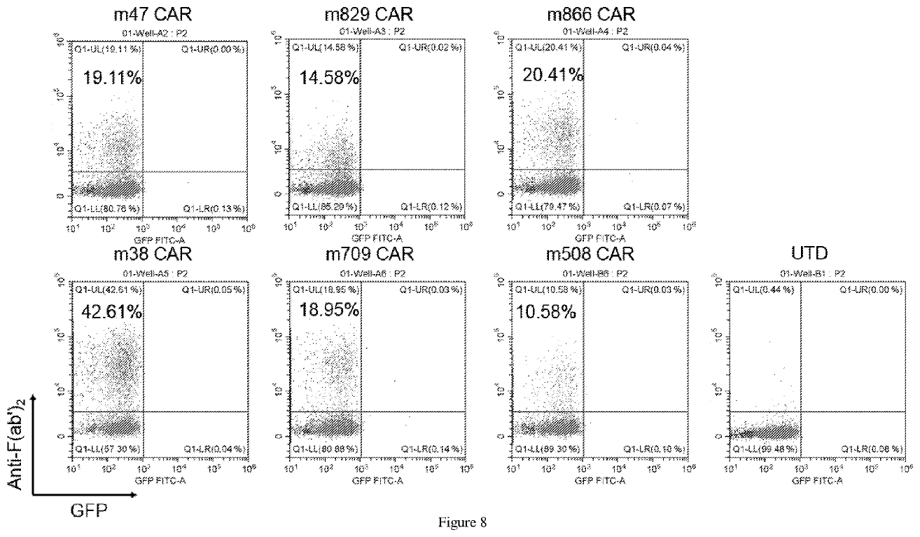

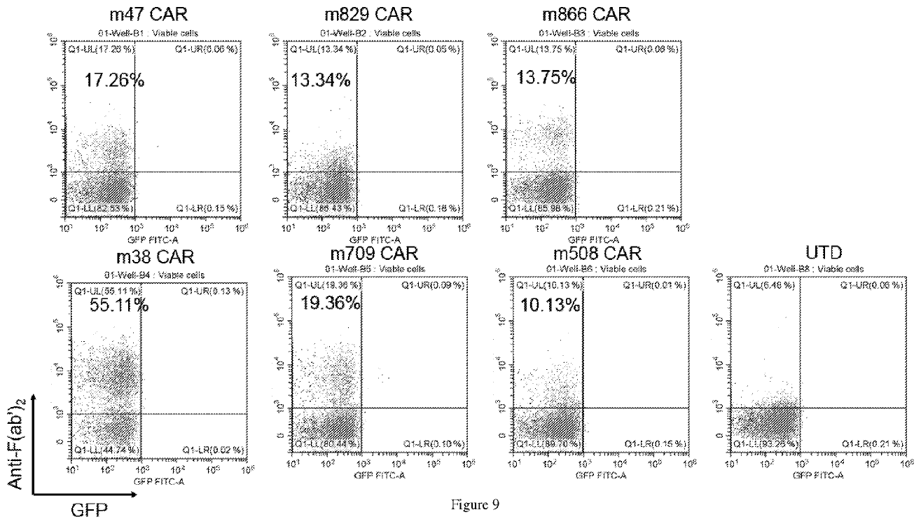

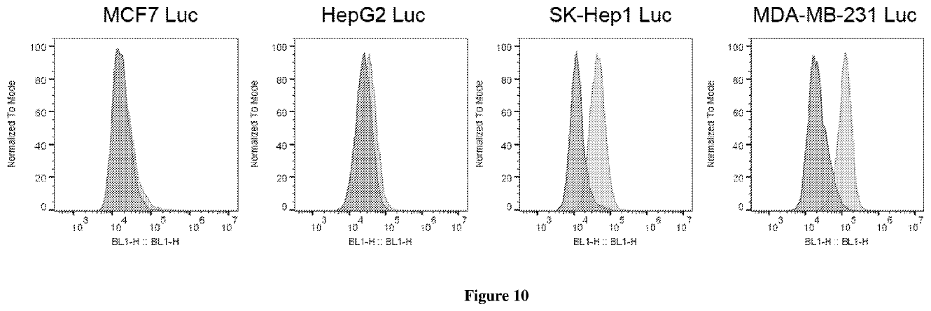

As a second screening step, we expressed m38 CAR, m47 CAR, m508 CAR, m709 CAR, m829 CAR, and m866 CAR in primary T cells isolated from whole blood of healthy donors by Ficoll-Paque gradient centrifugation and magnetic bead CD3 negative selection (Stem Cell Technologies). CAR expression in primary T cells was confirmed by flow cytometry using Alexa Fluor 647-conjugated goat anti-mouse IgG F(ab′)₂ antibodies. We screened cytotoxic potential across multiple donors to account for donor-to-donor variation. We confirmed ROR1 CAR expression both in donor ND22 (FIG. 8) and donor ND23 (FIG. 9). We then tested cytotoxicity against the following cell lines: MCF7, HepG2, SK-Hep-1 and MDA-MB-231, which express varying levels of ROR1 (FIG. 10). FIG. 10 shows varied ROR1 expression profile, wherein MCF7 represents ROR1-negative cells, HepG2 represents low ROR1-expressing cells, SK-Hep-1 represents medium ROR1-expressing cells, and MDA-MB-231 represents high ROR1-expressing cells. Dark gray histograms show the unlabeled control cells, while anti-ROR1 labeled cells are shown as light gray histograms.

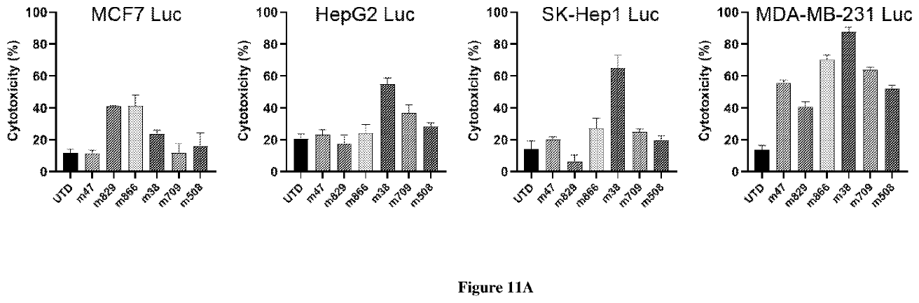

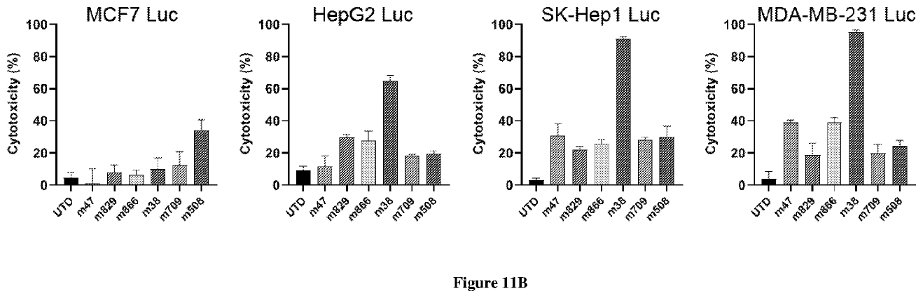

All cell lines used in this assay were luciferase positive. Percent cytotoxicity was determined as a decrease in bioluminescence relative to untreated control samples (UTD). The effector cells and target cells were 1:1 co-cultured overnight. The cytotoxicity assays of different CAR-T cells from different donors against MCF7, HepG2, SK-Hep-1, and MDA-MB-231 luciferase-expressing target cells are shown in FIGS. 11A-11B. It can be seen from FIGS. 11A-11B that the ROR1 scFv CARs from different donors have varied cytotoxicity against MCF7, HepG2, SK-Hep-1, and MDA-MB-231 target cells.

2.2. Validation of Humanized ROR1 CAR Candidates with Hu709 Scfv

Based on the results of the NFAT-luciferase reporter assays and cytotoxicity assays, we elected to proceed with humanization of m709. We generated three humanized 709 variants from two humanized VH and two humanized VL sequences: hu709 VH2VL2, hu709 VH4VL1, hu709 VH4VL2. We named the CAR constructs derived from hu709 variants as RC005a, RC005b, and RC005c, respectively. The three humanized CAR constructs were structured as shown in FIG. 1.

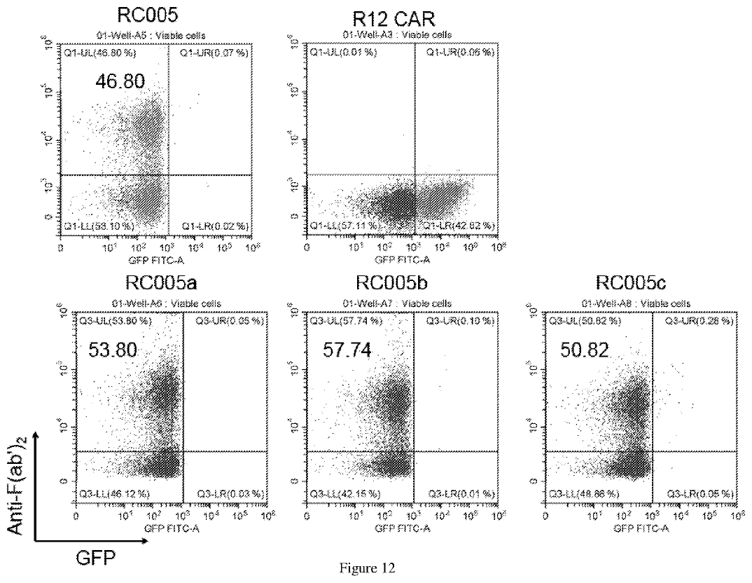

To validate the humanized ROR1 CAR variants, we performed an NFAT luciferase reporter assay using Jurkat cells transduced to express RC005 (i.e. m709 CAR), RC005a, RC005b, or RC005c. CAR expression was confirmed post electroporation using AF647-conjugated goat anti-human F(ab′)₂ antibodies (Jackson ImmunoResearch) or using AF647-conjugated goat anti-human F(ab′)2 antibodies for the original m709 variant (FIG. 12). In FIG. 12, a clinical-stage ROR1 targeting R12 CAR (synthesized by Genscript) was used as a benchmark control. It can be seen that the percentage of CAR positive Jurkat cells was found to be between 45-60% for all of the CAR variants assessed, indicating good construct expression on the surface of NFAT luciferase reporter cells.

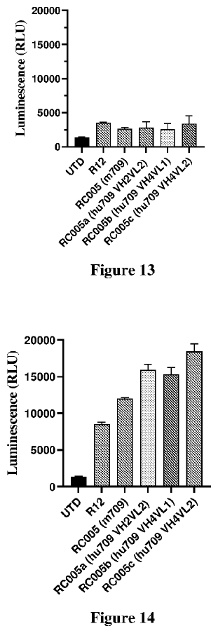

CAR-positive Jurkat Cells were cultured 1:1 overnight with ROR1-negative MCF7 cells (FIG. 13) or ROR1-positive SK-Hep-1 cells (FIG. 14). Luciferase activity was determined using NeoLite luciferase substrate and read out using a SpectraMax plate reader. It can be seen from FIGS. 13-14 that NFAT-driven luciferase expression was higher in our humanized 709 CAR variants than both the m709 CAR and the R12 CAR benchmark.

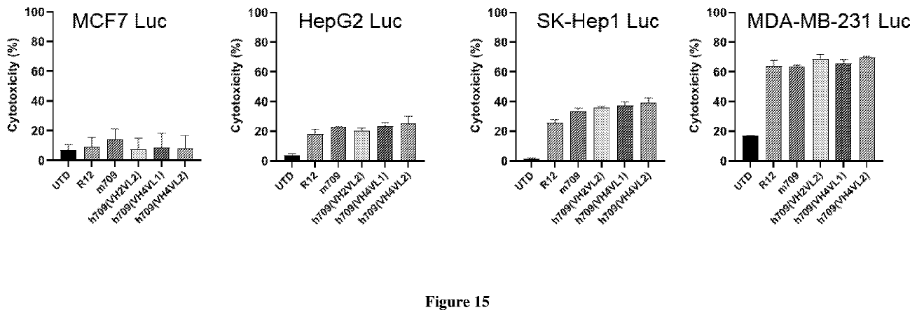

Once we had validated CAR function in Jurkat NFAT-luciferase reporter cells, we next wanted to determine the cytotoxic potential of the humanized CAR variants. Primary T cells derived from healthy donors were transduced by electroporation using the PiggyBac vector (non-EGFP plasmid, synthesized by Genscript) to express the ROR1 CARs and then used in cytotoxicity assays against ROR1-positive and ROR1-negative cell lines. We performed cytotoxicity assays against the luciferase-expressing target cell lines MCF7, HepG2, SK-Hep-1, and MDA-MB-231 using T cells derived from healthy donor ND22 (FIG. 15). To account for donor-to-donor differences, we repeated the cytotoxicity assays against the luciferase-expressing target cell lines MCF7, HepG2, SK-Hep-1, and MDA-MB-231 using T cells derived from healthy donor ND19 (FIG. 16). It can be seen from FIGS. 15-16 that our lead candidate ROR1 CARs (m709, h709(VH2VL2), h709(VH4VL1), h709(VH4VL2)) show equal cytotoxicity against MCF7, HepG2, SK-Hep-1, and MDA-MB-231 with Benchmark Control R12.

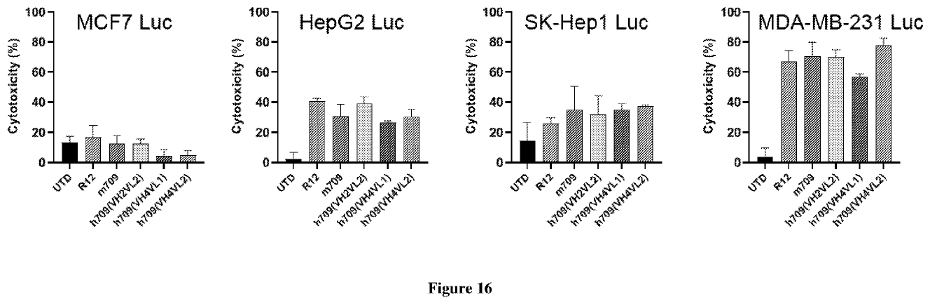

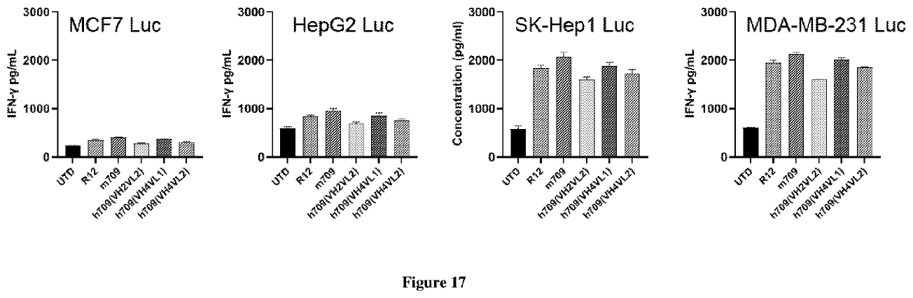

Additionally, we used supernatant harvested from the healthy donor ND19 CAR-T cytotoxicity assay to determine CAR-T cell cytokine secretion. One common readout of CAR T cell activation is IFN-γ. We used ELISA to determine IFN-γ secretion by CAR-T cells co-cultured 1:1 overnight with MCF7, HepG2, SK-Hep-1, or MDA-MB-231 cells (FIG. 17). IFN-γ secretion against the ROR1 positive cell line MDA-MB-231 was the highest. These data indicate that our humanized 709 CAR variants were as effective as the R12 CAR benchmark control against ROR1-positive target cells.

3. ROR1 CAR Candidates with Other Humanized ROR1 Scfv (e.g. Anti-ROR1 Clone 1720)

We named the CAR constructs derived from anti-ROR1 clone 1720 variant as 1720. In this example, we also compared CAR construct 1720 to CAR constructs R12 and RC005c (hu709 VH4VL2).

Peripheral blood mononuclear cells (PBMCs) purchased from AllCells were marked with microbeads through a CD3 MicroBeads human-lyophilized Kit (purchased from Miltenyi Biotech). CD3+T lymphocytes with high purity were selected, with a proportion of CD3 positive T cells over 95%. The purified T cells were activated and proliferated using a human CD3CD28 T cell activator (Dynabeads Human T-Activator CD3/CD28, Thermo Fisher, 11132D).

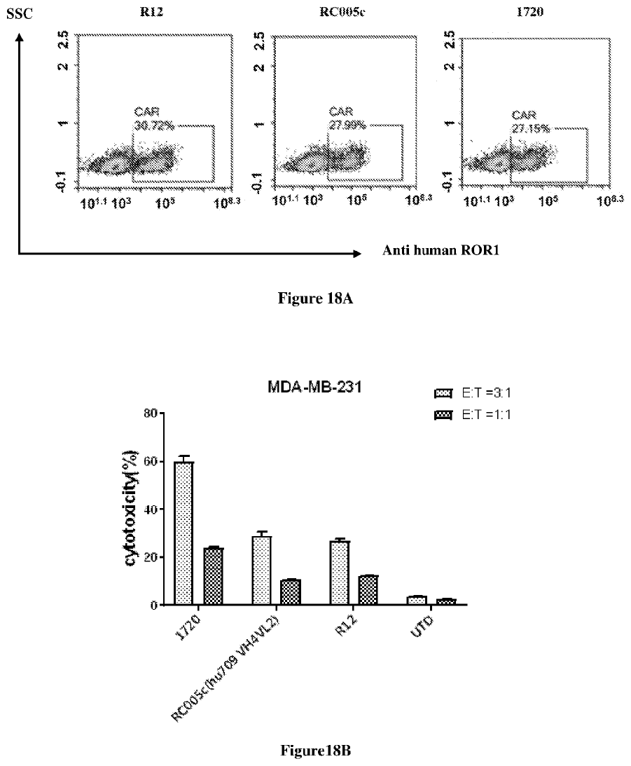

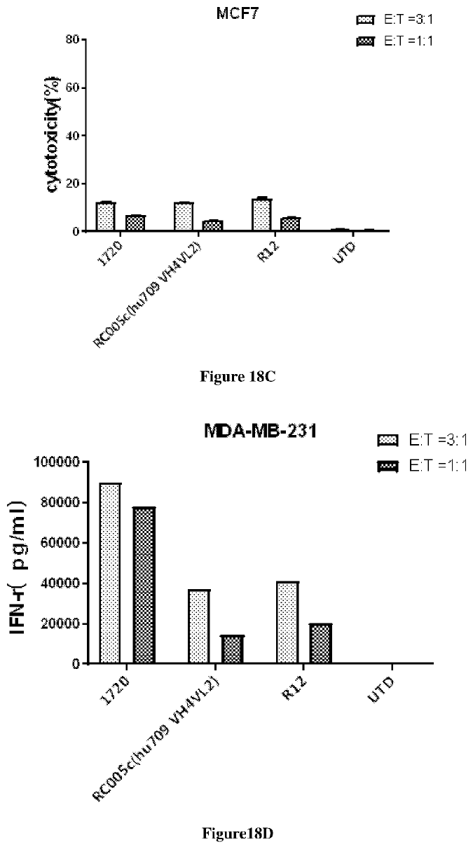

To screen for CAR activity, the above-obtained T cells were electroporated with PiggyBac plasmids (shown in FIG. 3) with the CAR cassette as well as PiggyBac Transposon mRNA. CAR expression in primary T cells was confirmed by flow cytometry using Biotinylated Human ROR1 Protein, His, Avitag (Acro biosystem) (FIG. 18A). From FIG. 18A, it can be seen that the CAR Positive rate for R12, RC005c (hu709 VH4VL2) and 1720 are 30.72%, 27.99%, and 27.15% respectively.

To validate the humanized ROR1 CAR variants, we tested cytotoxicity against the following cell lines: MDA-MB-231 cells (BeiNa BioTech) (FIG. 18B) and MCF-7 cells (BeiNa BioTech) (FIG. 18C). In this example, high ROR1-expressing cells MDA-MB-231 were used as target cells, ROR1-negative MCF-7 cells were used as negative target cells and ROR1 CAR-T cells were used as effector cells according to different E:T (effector cell:target cell) ratios. The results of in vitro experiments (FIG. 18B-FIG. 18 C) show that when co-culturing R12 ROR1 CAR-T (Benchmark), RC005c CAR-T, and 1720 CAR-T with MDA-MB-231/MCF-7 cells for 24h, the efficiency of killing tumor cells can reach 20%-100% at 24h (Table 11-12).

At the same time, the CAR-T specific response was evaluated by detecting the content of cytokines (IFN-gamma) in the supernatant of the culture medium. When co-culturing R12 ROR1 CAR-T (Benchmark), RC005c CAR-T, and 1720 CAR-T with MDA-MB-231/MCF-7 cells for 24h, IFN-gamma cytokine released in the co-culture supernatant was consistent with the killing test results (FIG. 18D-FIG. 18 E, Tables 13-14).

It can be seen from FIGS. 18B and 18D that T cells with 1720 CAR show higher cytotoxicity against MDA-MB-231 cells compared to Benchmark Control R12, and higher IFN-γ secretion.

TABLE 11
Results of in vitro experiments on specific killing of MDA-MB-
231 cancer cell line by ROR1 CAR-T cells with different scFv

Effector to Target ratio

| Specific lysis (%) | 3:1 | 1:1 |
|---|---|---|
| 1720 | 60.13 | 24.18 |
| RC005c (hu709 VH4VL2) | 32.62 | 10.57 |
| R12 | 28.87 | 12.63 |
| UTD | 3.84 | 2.57 |

TABLE 12
Results of in vitro experiments on specific killing of MCF-7
cancer cell line by ROR1 CAR-T cells with different scFv

Effector to Target ratio

| Specific lysis (%) | 3:1 | 1:1 |
|---|---|---|
| 1720 | 12.96 | 6.19 |
| RC005c(hu709 VH4VL2) | 12.40 | 4.46 |
| R12 | 12.37 | 5.79 |
| UTD | 0.88 | 0.20 |

TABLE 13
IFN-gamma cytokine release in the supernatant of different scFv
ROR1 CAR-T co-cultured with MDA-MB-231 cancer cell line

| | |
|---|---|
| IFN-gamma cytokine release | Effector to Target ratio |

| (pg/mL) | 3:1 | 1:1 |
|---|---|---|
| 1720 | 89498.73 | 78295.66 |
| RC005c(hu709 VH4VL2) | 36808.62 | 14581.76 |
| R12 | 40568.98 | 20520.48 |
| UTD | 800.52 | 653.83 |

TABLE 14
IFN-gamma cytokine release in the supernatant of different
scFv ROR1 CAR-T co-cultured with MCF-7 cancer cell line

| | |
|---|---|
| IFN-gamma cytokine release | Effector to Target ratio |

| (pg/mL) | 3:1 | 1:1 |
|---|---|---|
| 1720 | 8273.01 | 2850.73 |
| RC005c(hu709 VH4VL2) | 4461.64 | 1368.29 |
| R12 | 6682.03 | 2556.41 |
| UTD | 385.99 | 411.50 |

### Example 2: Humanization of Human CD19-Specific Chimeric Antigen Receptor

1. Design of Murine scFv CAR Candidates

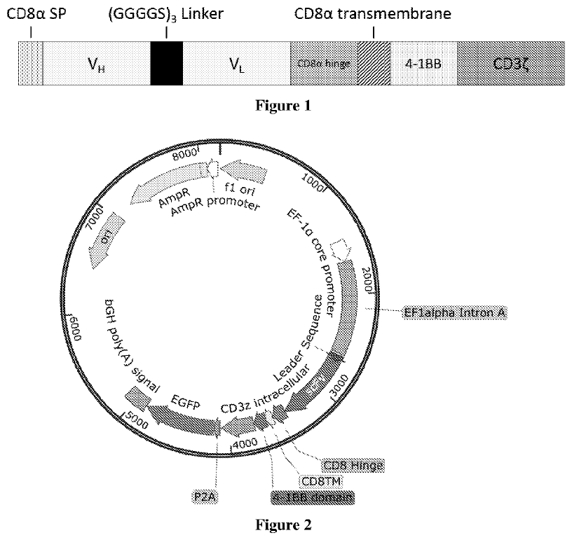

We designed 16 humanized anti-CD19 CAR variants (LS008a-LS008p) based on four humanized variants of the FMC63 heavy chain and four humanized variants of the FMC63 light chain. All CAR constructs were designed according to the following structure: CD8α SP-VH-(GGGGS)₃linker-VL-CD8α hinge domain CD8α transmembrane domain-4-1BB intracellular domain-CD3ζ intracellular domain (FIG. 1). In these constructs we used EGFP as an additional marker of CAR expression. EGFP was co-expressed via a P2A sequence at the 3′ end of the CAR sequence. The plasmid structure is shown simply in FIG. 2 (with EGFP). And in the present application document, “EGFP” and “GFP” are interchangeable because they share the same amino acid sequence shown in SEQ ID NO. 67.

2. Selection of One Murine scFv CAR Candidate for Humanization and Humanized scFv CAR Candidates

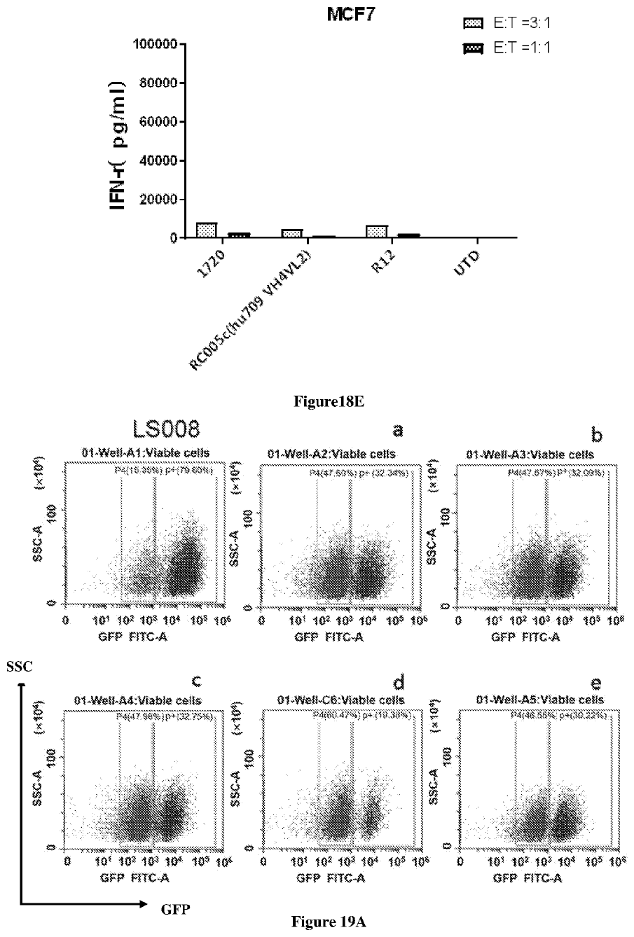

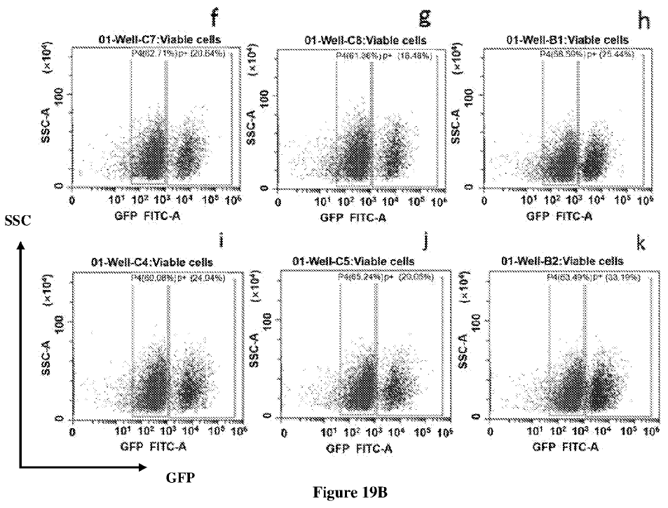

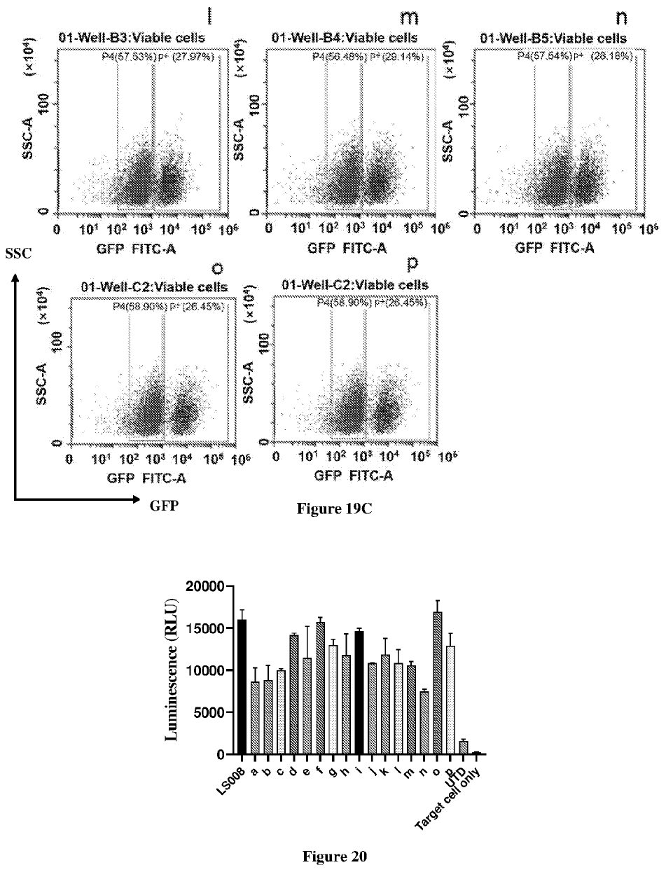

To screen the humanized CD19 CAR clones, Jurkat NFAT-luciferase reporter cells were transduced by electroporation to express the CAR variants. CAR expression and integration into the host cell genome were confirmed by flow cytometry. As previously mentioned, we included co-expression of EGFP on the PiggyBac CAR transposon to facilitate analysis of the CAR variants (FIG. 19A-19C). In FIG. 19 A-19C, the SSC of ordinate is a relative measure of cellular complexity, LS008 is a murine FMC63 scFv CAR (synthesized by Genscript) and used as a control, and a-p are humanized CD19 CAR variants LS008a-LS008p. It can be seen that all the humanized CAR variants a-p can be expressed on the surface of NFAT luciferase reporter cells.

To test the capacity of each humanized CD19 CAR variant to activate T cells, Jurkat NFAT-luciferase reporter cells expressing the CARs were cultured 1:1 with CD19-positive Raji cells overnight and then the luciferase activity was determined using NeoLite luciferase substrate (FIG. 20) and bioluminescence was read out using a SpectraMax plate reader. In FIG. 20, UTD represents untransduced parental Jurkat NFAT-luciferase reporter cells. We found that all of the humanized CD19 CAR variants LS008a-LS008p (represented by a-p in FIG. 20) were able to activate the CAR-expressing Jurkat cells in the presence of CD19-expressing target cells.

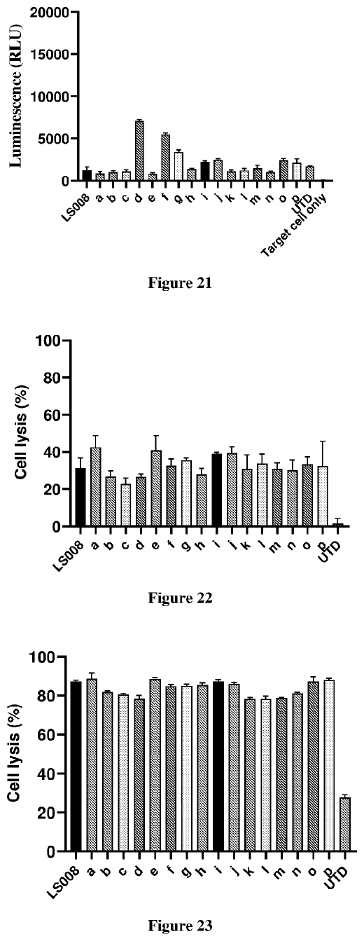

To determine baseline CAR activity or off-target activation, we repeated the luciferase assay with K562 CD19-negative target cells. Again, CAR-expressing Jurkat cells were cultured 1:1 overnight with the target cells and NFAT-driven luciferase expression was determined using NeoLite substrate (FIG. 21). By culturing with CD19-negative target cells, we were able to determine that most of the humanized variants had low off-target NFAT activity. However, we noted that humanized variants LS008d (huFMC63 VH1VL4 CAR) and LS008f (huFMC63 VH2VL2 CAR) had relatively high background NFAT activity.

3. Validation of Humanized scFv CAR Candidates

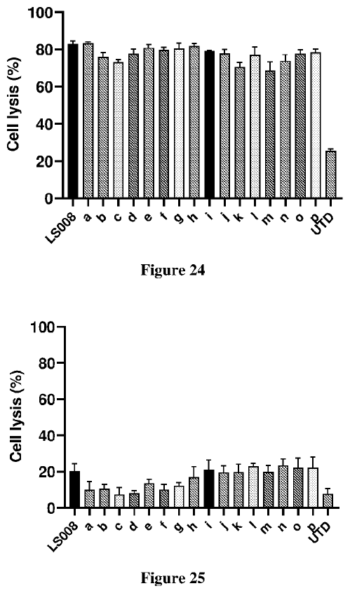

To evaluate the cytotoxic potential of the humanized FMC63 CD19 CAR variants, we performed flow cytometry-based cytotoxicity assays against the following CD19-positive cell lines: Raji, Jeko-1, and Nalm6; as well as the CD19-negative cell line K562. Specific lysis of the target cells was compared to the LS008 control and untransduced T cells (i.e. UTD). CAR T cells and target cells were co-cultured for 24 hours prior to FACS readout. Target cells were stained with cell trace dye and quantified to determine percent decrease in tumor cells relative to the untreated control. We observed humanized CD19 CAR variants (represented by a-p) have ˜30-40% antigen-specific cytotoxicity against Raji cells (FIG. 22). Against Jeko-1 (FIG. 23) cells and Nalm6 cells (FIG. 24), we observed higher cytotoxicity (˜80-90%). We did observe some background cytotoxicity against K562 cells in humanized clones expressing the huFMC63 VH3 and huFMC63 VH4 domains (i.e. LS008i-p), as well as in the LS008 (FIG. 25). From FIGS. 22-25, it can be seen that the CD19 CARs of the present application have cytotoxicity against CD19-positive Raji, Jeko-1 and Nalm6 cells, and LS008a shows enhanced specific cytotoxicity against Raji cells and lower non-specific cytotoxicity against K562 cells among all anti-CD19 CAR variants.

Based on both the cytotoxicity data and the NFAT data, we decided to proceed with further analysis of humanized anti-CD19 CAR variant LS008a.

### Example 3: Design of Human-CD19/Human-ROR1 Targeting Dual CAR Platform

1. Design Endocytic CAR Constructs that Contain SEZ6L2 tm Jm

SEZ6L2 is characterized by the presence of two endosomal-targeting consensus sequences in its c-terminal region. We designed a CAR construct containing a SEZ6L2 transmembrane domain and a SEZ6L2 juxtamembrane domain to solve the CAR-T safety problem by providing stealth CAR for reducing cytotoxicity towards normal cells.
1) ROR1scFv(hu709 VH4VL2)-CD8Hinge-SEZ6L2 tm jm-CD3Q
2) CD19 scFv(FMC63)-CD8Hinge-SEZ6L2 tm jm-CD3ζ

### 2. Design of CD19-ROR1 Dual CAR

Antigen escape is known to be a problem in CD19-targeted CAR T cell therapies. That is, selective pressure on the CD19 antigen results in downregulation of CD19 by tumor cells and ultimately in disease relapse from the CD19-negative tumor cells. Targeting two tumor-associated antigens decreases the likelihood of antigen escape by the target cells. Given that CD19 and ROR1 are co-expressed in a large proportion of leukemia, lymphoma, and myeloma subsets, we wanted to establish a dual-targeting CAR platform for both antigens simultaneously. To do this, we established a bicistronic expression vector to express both CD19 and ROR1 CARs in tandem from the same cassette (FIG. 26).

To set up the dual CAR construct, we initially used the murine FMC63 sequence for CD19 and the humanized 709 variant 3 (hu709 VH4VL2). We set up two versions of the construct, RC025 and RC026. RC025 comprises a dominant CD19 CAR with CD8α transmembrane domain and 4-1BB intracellular domain and a nondominant ROR1 lacking the 4-1BB domain and with the SEZ6L2 transmembrane and juxtamembrane domain (i.e. SEZ6L2 tm jm), which limits surface stability. In RC026, ROR1 is the dominant CAR and CD19 is the nondominant CAR. As expression of the second CAR cannot be detected at the surface by conventional flow cytometry antibody staining, we used EGFP as a marker of nondominant CAR expression.

Specifically, the RC025 CAR comprises from N-terminal to C-terminal: CD19 scFv(FMC63)-CD8Hinge-CD8 tm-4-1BB-CD3ζ-P2A-ROR1scFv(hu709)-CD8Hinge-SEZ6L2 tm jm-CD3ζ-(-GS linker-GFP; and the RC026 CAR comprises from N-terminal to C-terminal: ROR1scFv(hu709)-CD8Hinge-CD8 tm-4-1BB-CD3ζ-P2A-CD19 scFv(FMC63)-CD8Hinge-SEZ6L2 tm jm-CD3ζ-(-GS linker-GFP.

### 3. Validation of CD19-ROR1 Dual CAR

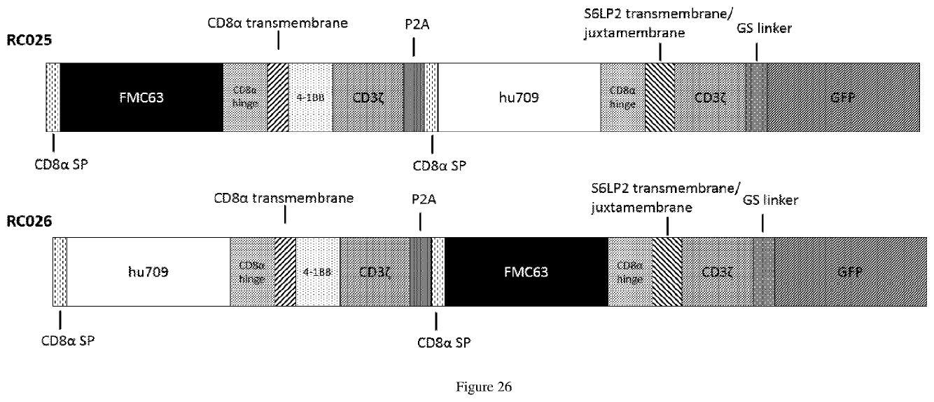

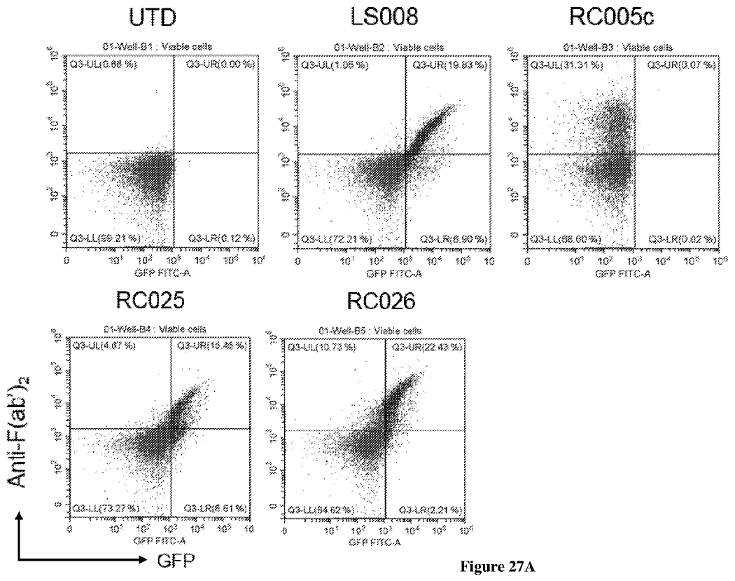

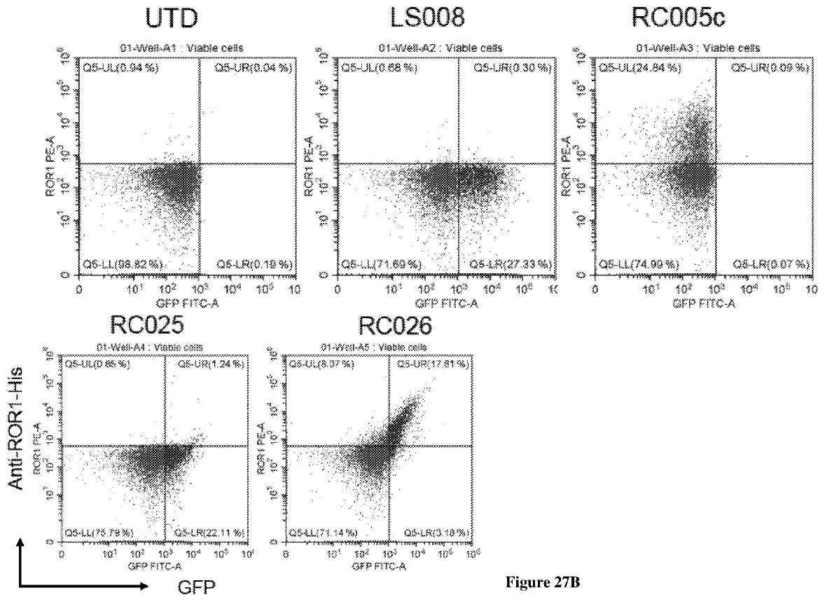

To test Dual CAR function, we transduced Jurkat NFAT-Luciferase reporter cells with the PiggyBac construct by electroporation and used flow cytometry to determine CAR expression 3 days after electroporation. In this assay we used both AF647-conjugated goat anti-mouse or anti-human F(ab′)₂, as well as recombinant human ROR1 (rhROR1-His), which was then detected using anti-his antibody. LS008 (murine anti-CD19 CAR) and RC005c (humanized anti-ROR1 CAR) were included as single CAR control constructs. As expected, we were able to detect CAR expression using anti-F(ab′)₂ in all of the samples tested-LS008, RC005c, RC025 (dual CAR), and RC026 (dual CAR)—but we were only able to observe rhROR1 labeling in RC005c and RC026, which have the dominant ROR1 CARs (FIG. 27A-27B). From FIG. 27A-27B, it can be seen that all CARs were expressed in Jurkat NFAT luciferase reporter cells.

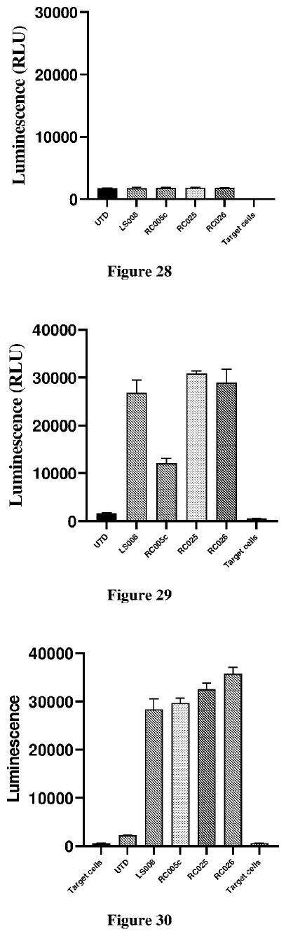

To further confirm Dual CAR functionality, we screened Jurkat NFAT-luciferase cells expressing LS008, RC005c, RC025, and RC026 against ROR1 positive and negative cell lines. Jurkat NFAT-luciferase cells and ROR1 and CD19 double negative K562 cells were co-cultured 1:1 overnight to determine background T cell activation. As expected, we observed very low NFAT reporter activity in response to the ROR1 and CD19-double negative cell line, K562 (FIG. 28). Importantly, we saw a high level of NFAT reporter activity in RC025 and RC026 relative to the LS008 control cells in response to 1:1 overnight co-culture with CD19-positive cell line Raji cells, indicating that the Dual CAR variants RC025 and RC026 were both responsive to CD19 antigen (FIG. 29). We also saw strong NFAT activity in all the CAR constructs tested in response to 1:1 overnight co-culture with ROR1/CD19 double positive cell line Jeko-1 (FIG. 30).

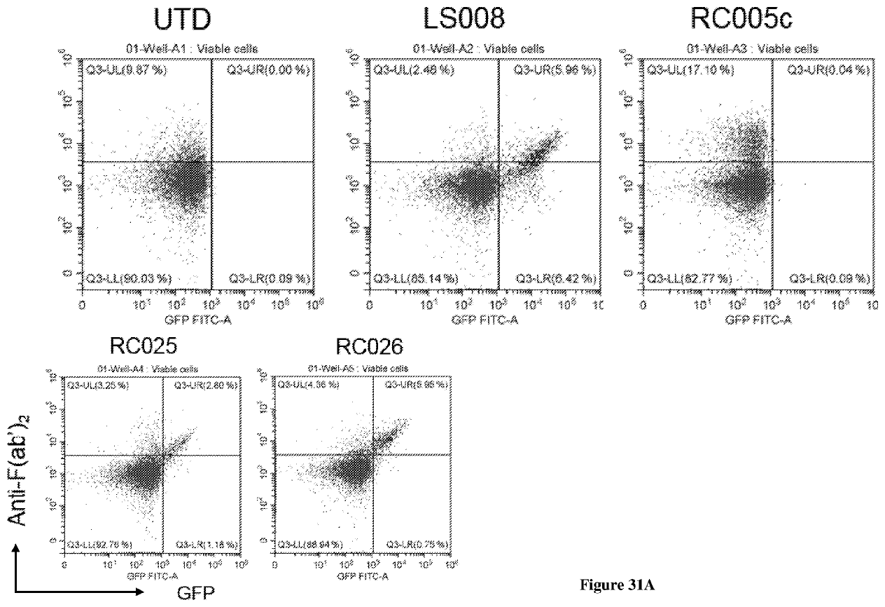

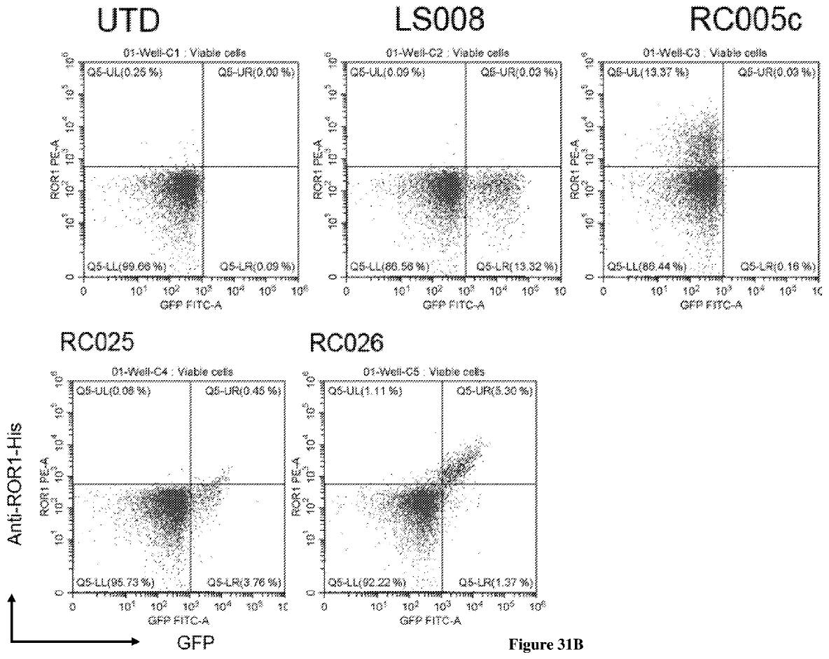

To further validate the dual CAR construct, we performed flow cytometry-based cytotoxicity assays against ROR1 and CD19 positive and negative cell lines. Primary T cells derived from healthy donors were transduced by electroporation to express LS008, RC005c, RC025, and RC026. CAR expression was confirmed 3 days after electroporation by flow cytometry by labeling the cells with AF647 conjugated anti-human or mouse F(ab′)₂ antibodies or rhROR1 (FIG. 31A-31B). From FIG. 31A-B, it can be seen that all CARs were expressed in Primary T cells.

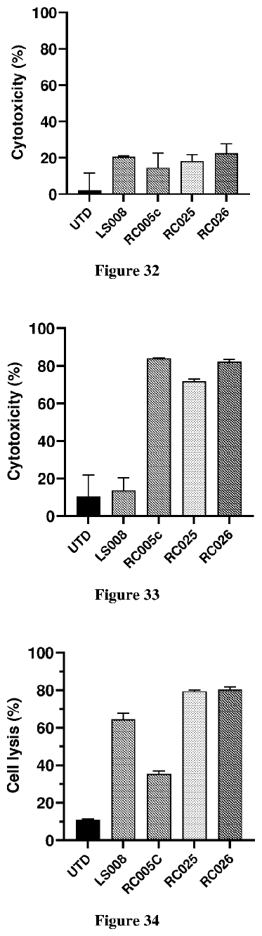

We tested our constructs against different cell lines. CAR T cells generated from healthy donor ND22 were co-cultured for 24 hours with CD19/ROR1 double negative cell line MCF7. The MCF7 target cells were engineered to express luciferase and percent cytotoxicity was calculated as the decrease in bioluminescence in the CAR T cell treatment groups relative to untreated control cells. We did observe some background cytotoxicity in LS008, RC005c, RC025, and RC026, but this was comparable among all the constructs tested (˜20%) (FIG. 32).

CAR T cells generated from healthy donor ND22 were co-cultured for 24 hours with the ROR1-positive cell line MDA-MB-231. The MDA-MB-231 target cells were engineered to express luciferase and percent cytotoxicity was calculated as the decrease in bioluminescence in the CAR T cell treatment groups relative to untreated control cells. RC005c (humanized anti-ROR1 CAR), RC025, and RC026 showed comparable levels of cytotoxicity (˜80%) against ROR1-positive MDA-MB-231 cells indicating target-specific lysis by the dominant and nondominant versions of the ROR1 CAR in RC025 and RC026. LS008 showed minimal cytotoxicity against MDA-MB-231 cells indicating that the cytotoxicity observed was attributable to the ROR1 CAR (FIG. 33).

CAR T cells generated from healthy donor ND22 were co-cultured for 24 hours with the ROR1/CD19 double positive cell line Jeko-1. This cytotoxicity assay was performed via flow cytometry. Jeko-1 cells were labeled with cell trace dye and target-specific lysis was calculated as the percent decrease in Jeko-1 cells in the treatment groups relative to the negative control group. We observed specific lysis of Jeko-1 cells by all the CAR constructs with the LS008 CD19 single CAR outperforming the RC005c single CAR. Importantly, RC025 and RC026, the ROR1/CD19 dual CARs, showed comparable levels of cytotoxicity (˜80%) that were higher than either of the single CAR systems (FIG. 34). These results indicate that there is a synergy between the ROR1 CAR and CD19 CAR in killing double positive target cells.

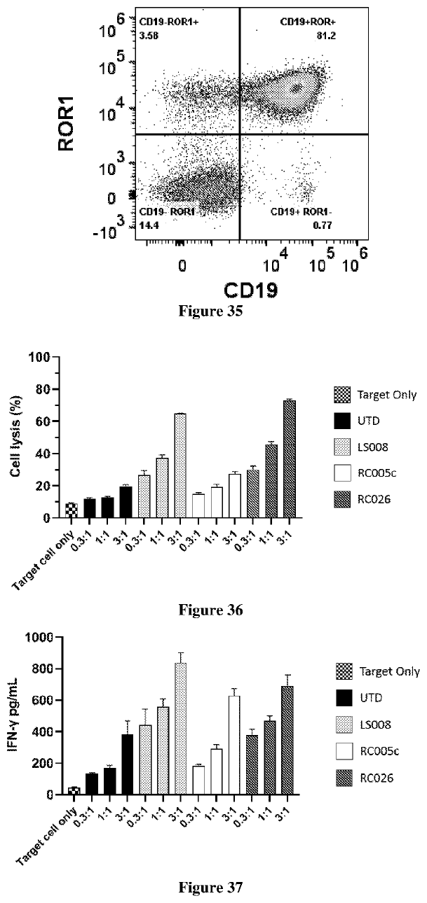

We next tested the dual CAR construct against primary patient tumors. In this assay, DLBCL tumor samples were obtained from patients and assessed for CD19 and ROR1 expression by flow cytometry (FIG. 35). Although ˜14% of these cells were determined to be negative for CD19 and ROR1, the majority of tumor cells (˜86%) were found to be positive for a combination of ROR1 and CD19, with ˜81% of the cells double positive for both antigens. We performed a flow cytometry-based killing assay on the patient-derived tumor cells using LS008, RC005c, and RC026. CAR T cells generated from a healthy donor (ND19) were used to determine cytotoxicity against patient-derived DLBCL tumor cells. In this assay, we used E:T ratios 3:1, 1:1, and 0.3:1. In this assay, patient-derived tumor cells were labeled using cell trace dye and quantified under each condition. Specific lysis was determined as a decrease in the fraction of live cells between CAR T treated tumor cells and an untreated control population. We found that RC026 was more effective at driving lysis of the tumor cell population, but that RC005c performed only slightly above the UTD cell baseline (FIG. 36). Supernatant collected from the cytotoxicity assay was assayed by ELISA for IFN-γ. We found that LS008 yielded the highest levels of IFN-γ secretion, while RC026, which had higher levels of cytotoxicity, still had lower levels of cytokine release (FIG. 37). RC005c had the lowest levels of cytokine release, which corresponded well with the lower levels of cytotoxicity. Taken together these data suggest that dual-targeting ROR11CD19 CAR constructs may have improved therapeutic impact against double positive tumors.

### OTHER EMBODIMENTS

It is to be understood that while the invention has been described in conjunction with the detailed description thereof, the foregoing description is intended to illustrate and not limit the scope of the disclosure, which is defined by the scope of the appended claims. Other aspects, advantages, and modifications are within the scope of the following claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of these various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A chimeric antigen receptor (CAR) comprising,
(1) an extracellular ligand-binding domain comprising scFv specifically binding to Receptor tyrosine kinase-like Orphan Receptor 1 (ROR1);
(2) a transmembrane domain; wherein preferably, the transmembrane domain is CD8 transmembrane domain; or
a transmembrane (tm) linking juxtamembrane (jm) domain, wherein the transmembrane linking juxtamembrane domain comprises a Seizure 6-like Protein 2 (SEZ6L2) transmembrane domain and a SEZ6L2 juxtamembrane domain; and
(3) an intracellular domain; wherein preferably, the intracellular domain comprises a signaling domain; more preferably, the signaling domain comprises one or more signaling domains selected from the group consisting of a 4-1BB signaling domain, a CD28 signaling domain and a CD3ζ signaling domain;
wherein the scFv specifically binding to ROR1 comprises:
HCDR1 shown in SEQ ID NO.: 10, HCDR2 shown in SEQ ID NO.: 11, HCDR3 shown in SEQ ID NO.: 12, LCDR1 shown in SEQ ID NO.: 27, LCDR2 shown in SEQ ID NO.: 28 and LCDR3 shown in SEQ ID NO.: 29;
HCDR1 shown in SEQ ID NO.: 1, HCDR2 shown in SEQ ID NO.: 2, HCDR3 shown in SEQ ID NO.: 3, LCDR1 shown in SEQ ID NO.: 18, LCDR2 shown in SEQ ID NO.: 19 and LCDR3 shown in SEQ ID NO.: 20;
HCDR1 shown in SEQ ID NO.: 4, HCDR2 shown in SEQ ID NO.: 5, HCDR3 shown in SEQ ID NO.: 6, LCDR1 shown in SEQ ID NO.: 21, LCDR2 shown in SEQ ID NO.: 22 and LCDR3 shown in SEQ ID NO.: 23;
HCDR1 shown in SEQ ID NO.: 7, HCDR2 shown in SEQ ID NO.: 8, HCDR3 shown in SEQ ID NO.: 9, LCDR1 shown in SEQ ID NO.: 24, LCDR2 shown in SEQ ID NO.: 25 and LCDR3 shown in SEQ ID NO.: 26;
HCDR1 shown in SEQ ID NO.: 10, HCDR2 shown in SEQ ID NO.: 11, HCDR3 shown in SEQ ID NO.: 12, LCDR1 shown in SEQ ID NO.: 27, LCDR2 shown in SEQ ID NO.: 28 and LCDR3 shown in SEQ ID NO.: 29;
HCDR1 shown in SEQ ID NO.: 13, HCDR2 shown in SEQ ID NO.: 14, HCDR3 shown in SEQ ID NO.: 15, LCDR1 shown in SEQ ID NO.: 30, LCDR2 shown in SEQ ID NO.: 31 and LCDR3 shown in SEQ ID NO.: 32;
HCDR1 shown in SEQ ID NO.: 10, HCDR2 shown in SEQ ID NO.:16, HCDR3 shown in SEQ ID NO.: 17, LCDR1 shown in SEQ ID NO.: 33, LCDR2 shown in SEQ ID NO.: 34 and LCDR3 shown in SEQ ID NO.: 35;
HCDR1 shown in SEQ ID NO.: 10, HCDR2 shown in SEQ ID NO.:11, HCDR3 shown in SEQ ID NO.: 12, LCDR1 shown in SEQ ID NO.: 27, LCDR2 shown in SEQ ID NO.: 28 and LCDR3 shown in SEQ ID NO.: 29; or
HCDR1 shown in SEQ ID NO.: 83, HCDR2 shown in SEQ ID NO.:84, HCDR3 shown in SEQ ID NO.: 85, LCDR1 shown in SEQ ID NO.:86, LCDR2 shown in SEQ ID NO.: 87 and LCDR3 shown in SEQ ID NO.: 88.

2. The CAR of claim 1, wherein the scFv specifically binding to ROR1 comprises:
VH comprising an amino acid sequence that is at least about 95%, 96%, 97%, 98%, 99% or 100% identical to the amino acid sequence represented by SEQ ID NO.: 57 and VL comprising an amino acid sequence that is at least about 95%, 96%, 97%, 98%, 99% or 100% identical to the amino acid sequence represented by SEQ ID NO.: 59;
VH comprising an amino acid sequence that is at least about 95%, 96%, 97%, 98%, 99% or 100% identical to the amino acid sequence represented by SEQ ID NO.: 44 and VL comprising an amino acid sequence that is at least about 95%, 96%, 97%, 98%, 99% or 100% identical to the amino acid sequence represented by SEQ ID NO.: 50;
VH comprising an amino acid sequence that is at least about 95%, 96%, 97%, 98%, 99% or 100% identical to the amino acid sequence represented by SEQ ID NO.: 45 and VL comprising an amino acid sequence that is at least about 95%, 96%, 97%, 98%, 99% or 100% identical to the amino acid sequence represented by SEQ ID NO.:51;
VH comprising an amino acid sequence that is at least about 95%, 96%, 97%, 98%, 99% or 100% identical to the amino acid sequence represented by SEQ ID NO.: 46 and VL comprising an amino acid sequence that is at least about 95%, 96%, 97%, 98%, 99% or 100% identical to the amino acid sequence represented by SEQ ID NO.:52;
VH comprising an amino acid sequence that is at least about 95%, 96%, 97%, 98%, 99% or 100% identical to the amino acid sequence represented by SEQ ID NO.:47 and VL comprising an amino acid sequence that is at least about 95%, 96%, 97%, 98%, 99% or 100% identical to the amino acid sequence represented by SEQ ID NO.:53;
VH comprising an amino acid sequence that is at least about 95%, 96%, 97%, 98%, 99% or 100% identical to the amino acid sequence represented by SEQ ID NO.: 48 and VL comprising an amino acid sequence that is at least about 95%, 96%, 97%, 98%, 99% or 100% identical to the amino acid sequence represented by SEQ ID NO.: 54;
VH comprising an amino acid sequence that is at least about 95%, 96%, 97%, 98%, 99% or 100% identical to the amino acid sequence represented by SEQ ID NO.: 49 and VL comprising an amino acid sequence that is at least about 95%, 96%, 97%, 98%, 99% or 100% identical to the amino acid sequence represented by SEQ ID NO.:55;
VH comprising an amino acid sequence that is at least about 95%, 96%, 97%, 98%, 99% or 100% identical to the amino acid sequence represented by SEQ ID NO.:56 and VL comprising an amino acid sequence that is at least about 95%, 96%, 97%, 98%, 99% or 100% identical to the amino acid sequence represented by SEQ ID NO.:59;
VH comprising an amino acid sequence that is at least about 95%, 96%, 97%, 98%, 99% or 100% identical to the amino acid sequence represented by SEQ ID NO.:57 and VL comprising an amino acid sequence that is at least about 95%, 96%, 97%, 98%, 99% or 100% identical to the amino acid sequence represented by SEQ ID NO.:58; or
VH comprising an amino acid sequence that is at least about 95%, 96%, 97%, 98%, 99% or 100% identical to the amino acid sequence represented by SEQ ID NO.:81 and VL comprising an amino acid sequence that is at least about 95%, 96%, 97%, 98%, 99% or 100% identical to the amino acid sequence represented by SEQ ID NO.: 82.

3. The CAR of claim 1, wherein the SEZ6L2 transmembrane-juxtamembrane domain comprises an amino acid sequence that is at least about 95%, 96%, 97%, 98%, 99% or 100% identical to the amino acid sequence represented by SEQ ID NO.: 66.

4. The CAR of claim 1, wherein the CAR comprises from N-terminal to C-terminal:
1) ROR1 scFv-CD8Hinge-CD8 tm-4-1BB-CD3ζ; or
2) ROR1 scFv-CD8Hinge-SEZ6L2 tm jm-CD3ζ;
wherein preferably, the N-terminal of the CAR further contains a leader sequence;
wherein preferably, the leader sequence comprises an amino acid sequence that is at least about 95%, 96%, 97%, 98%, 99% or 100% identical to the amino acid sequence represented by SEQ ID NO. 61;
the CD8Hinge comprises an amino acid sequence that is at least about 95%, 96%, 97%, 98%, 99% or 100% identical to the amino acid sequence represented by SEQ ID NO.: 62,
the CD8tm comprises an amino acid sequence that is at least about 95%, 96%, 97%, 98%, 99% or 100% identical to the amino acid sequence represented by SEQ ID NO.: 63,
the 4-1BB intracellular domain comprises an amino acid sequence that is at least about 95%, 96%, 97%, 98%, 99% or 100% identical to the amino acid sequence represented by SEQ ID NO.: 64, and
the CD3ζ intracellular domain comprises an amino acid sequence that is at least about 95%, 96%, 97%, 98%, 99% or 100% identical to the amino acid sequence represented by SEQ ID NO.: 65.

5. A dual CAR comprising: the CAR of claim 1, and
a second CAR comprising:
(1) an extracellular ligand-binding domain comprising scFv specifically binding to a predetermined antigen; wherein the predetermined antigen is a tumor-associated antigen (TAA); more preferably, the TAA is selected from one or more of CEA, Claudin 18.2, CGC3, CD38, CD19, CD20, CD22, BCMA, CAIX, CD446, CD13, EGFR, EGFRvIII, EpCam, GD2, EphA2, HER1, HER2, ICAM-1, IL13Ra2, Mesothelin, MUC1, MUC16, PSCA, NY-ESO-1, MART-1, WT1, MAGE-A10, MAGE-A3, MAGE-A4, EBV, NKG2D, PD1, PD-L1, CD25, TL-2 and/or CD3;
(2) a transmembrane domain, wherein preferably, the transmembrane domain is CD8 transmembrane domain; or
a transmembrane (tm) linking juxtamembrane (jm) domain, wherein the transmembrane linking juxtamembrane domain comprises a Seizure 6-like Protein 2 (SEZ6L2) transmembrane domain and a SEZ6L2 juxtamembrane domain; and
(3) an intracellular domain; wherein preferably, the intracellular domain comprises a signaling domain; more preferably, the signaling domain comprises one or more signaling domains selected from the group consisting of a 4-1BB signaling domain, a CD28 signaling domain and a CD3ζ signaling domain;
wherein preferably, the first CAR targets ROR1 and the second CAR targets another antigen,
wherein preferably, the first CAR and the second CAR are linked by P2A.

6. The dual CAR of claim 5, wherein the TAA is CD19, and the CD19 scFv comprises:
HCDR1 shown in SEQ ID NO.: 37, HCDR2 shown in SEQ ID NO.: 38, HCDR3 shown in SEQ ID NO.: 39, LCDR1 shown in SEQ ID NO.: 41, LCDR2 shown in SEQ ID NO.: 42 and LCDR3 shown in SEQ ID NO.: 43; or
HCDR1 shown in SEQ ID NO.: 37 HCDR2 shown in SEQ ID NO.: 38, HCDR3 shown in SEQ ID NO.: 40, LCDR1 shown in SEQ ID NO.: 41, LCDR2 shown in SEQ ID NO.: 42 and LCDR3 shown in SEQ ID NO.: 43.

7. The dual CAR of claim 6, wherein the CD19 scFv comprises:
VH comprising an amino acid sequence that is at least about 95%, 96%, 97%, 98%, 99% or 100% identical to the amino acid sequence represented by SEQ ID NO.: 69 and VL comprising an amino acid sequence that is at least about 95%, 96%, 97%, 98%, 99% or 100% identical to the amino acid sequence represented by SEQ ID NO.: 70;
VH comprising an amino acid sequence that is at least about 95%, 96%, 97%, 98%, 99% or 100% identical to the amino acid sequence represented by SEQ ID NO.: 71 and VL comprising an amino acid sequence that is at least about 95%, 96%, 97%, 98%, 99% or 100% identical to the amino acid sequence represented by SEQ ID NO.: 75;
VH comprising an amino acid sequence that is at least about 95%, 96%, 97%, 98%, 99% or 100% identical to the amino acid sequence represented by SEQ ID NO.: 71 and VL comprising an amino acid sequence that is at least about 95%, 96%, 97%, 98%, 99% or 100% identical to the amino acid sequence represented by SEQ ID NO.:76;
VH comprising an amino acid sequence that is at least about 95%, 96%, 97%, 98%, 99% or 100% identical to the amino acid sequence represented by SEQ ID NO.: 71 and VL comprising an amino acid sequence that is at least about 95%, 96%, 97%, 98%, 99% or 100% identical to the amino acid sequence represented by SEQ ID NO.:77;
VH comprising an amino acid sequence that is at least about 95%, 96%, 97%, 98%, 99% or 100% identical to the amino acid sequence represented by SEQ ID NO.:71 and VL comprising an amino acid sequence that is at least about 95%, 96%, 97%, 98%, 99% or 100% identical to the amino acid sequence represented by SEQ ID NO.:78;
VH comprising an amino acid sequence that is at least about 95%, 96%, 97%, 98%, 99% or 100% identical to the amino acid sequence represented by SEQ ID NO.: 72 and VL comprising an amino acid sequence that is at least about 95%, 96%, 97%, 98%, 99% or 100% identical to the amino acid sequence represented by SEQ ID NO.: 75;
VH comprising an amino acid sequence that is at least about 95%, 96%, 97%, 98%, 99% or 100% identical to the amino acid sequence represented by SEQ ID NO.:72 and VL comprising an amino acid sequence that is at least about 95%, 96%, 97%, 98%, 99% or 100% identical to the amino acid sequence represented by SEQ ID NO.:76;
VH comprising an amino acid sequence that is at least about 95%, 96%, 97%, 98%, 99% or 100% identical to the amino acid sequence represented by SEQ ID NO.:72 and VL comprising an amino acid sequence that is at least about 95%, 96%, 97%, 98%, 99% or 100% identical to the amino acid sequence represented by SEQ ID NO.:77;
VH comprising an amino acid sequence that is at least about 95%, 96%, 97%, 98%, 99% or 100% identical to the amino acid sequence represented by SEQ ID NO.:72 and VL comprising an amino acid sequence that is at least about 95%, 96%, 97%, 98%, 99% or 100% identical to the amino acid sequence represented by SEQ ID NO.:78;
VH comprising an amino acid sequence that is at least about 95%, 96%, 97%, 98%, 99% or 100% identical to the amino acid sequence represented by SEQ ID NO.:73 and VL comprising an amino acid sequence that is at least about 95%, 96%, 97%, 98%, 99% or 100% identical to the amino acid sequence represented by SEQ ID NO.:75;
VH comprising an amino acid sequence that is at least about 95%, 96%, 97%, 98%, 99% or 100% identical to the amino acid sequence represented by SEQ ID NO.:73 and VL comprising an amino acid sequence that is at least about 95%, 96%, 97%, 98%, 99% or 100% identical to the amino acid sequence represented by SEQ ID NO.:76;
VH comprising an amino acid sequence that is at least about 95%, 96%, 97%, 98%, 99% or 100% identical to the amino acid sequence represented by SEQ ID NO.:73 and VL comprising an amino acid sequence that is at least about 95%, 96%, 97%, 98%, 99% or 100% identical to the amino acid sequence represented by SEQ ID NO.:77;
VH comprising an amino acid sequence that is at least about 95%, 96%, 97%, 98%, 99% or 100% identical to the amino acid sequence represented by SEQ ID NO.:73 and VL comprising an amino acid sequence that is at least about 95%, 96%, 97%, 98%, 99% or 100% identical to the amino acid sequence represented by SEQ ID NO.:78;
VH comprising an amino acid sequence that is at least about 95%, 96%, 97%, 98%, 99% or 100% identical to the amino acid sequence represented by SEQ ID NO.:74 and VL comprising an amino acid sequence that is at least about 95%, 96%, 97%, 98%, 99% or 100% identical to the amino acid sequence represented by SEQ ID NO.:75;
VH comprising an amino acid sequence that is at least about 95%, 96%, 97%, 98%, 99% or 100% identical to the amino acid sequence represented by SEQ ID NO.:74 and VL comprising an amino acid sequence that is at least about 95%, 96%, 97%, 98%, 99% or 100% identical to the amino acid sequence represented by SEQ ID NO.:76;
VH comprising an amino acid sequence that is at least about 95%, 96%, 97%, 98%, 99% or 100% identical to the amino acid sequence represented by SEQ ID NO.:74 and VL comprising an amino acid sequence that is at least about 95%, 96%, 97%, 98%, 99% or 100% identical to the amino acid sequence represented by SEQ ID NO.:77; or
VH comprising an amino acid sequence that is at least about 95%, 96%, 97%, 98%, 99% or 100% identical to the amino acid sequence represented by SEQ ID NO.:74 and VL comprising an amino acid sequence that is at least about 95%, 96%, 97%, 98%, 99% or 100% identical to the amino acid sequence represented by SEQ ID NO.:78.

8. The dual CAR of claim 5, wherein the dual CAR comprises, from N-terminal to C-terminal:
TAA scFv-CD8Hinge-CD8tm-4-1BB-CD3ζ-P2A-ROR1scFv-CD8Hinge-SEZ6L2 tm jm-CD3ζ; or
ROR1scFv-CD8Hinge-CD8tm-4-1BB-CD3ζ-P2A-TAA scFv-CD8Hinge-SEZ6L2 tm jm-CD3ζ; wherein
preferably, the N-terminal of the CAR further contains a leader sequence;
preferably, the C-terminal of the CAR further contains a P2A-EGFP sequence;
preferably, the leader sequence comprises an amino acid sequence that is at least about 95%, 96%, 97%, 98%, 99% or 100% identical to the amino acid sequence represented by SEQ ID NO. 61;
preferably, the CD8Hinge comprises an amino acid sequence that is at least about 95%, 96%, 97%, 98%, 99% or 100% identical to the amino acid sequence represented by SEQ ID NO.: 62;
the CD8tm comprises an amino acid sequence that is at least about 95%, 96%, 97%, 98%, 99% or 100% identical to the amino acid sequence represented by SEQ ID NO.: 63;
the 4-1BB intracellular domain comprises an amino acid sequence that is at least about 95%, 96%, 97%, 98%, 99% or 100% identical to the amino acid sequence represented by SEQ ID NO.: 64;
the CD3ζ intracellular domain comprises an amino acid sequence that is at least about 95%, 96%, 97%, 98%, 99% or 100% identical to the amino acid sequence represented by SEQ ID NO.: 65;
the SEZ6L2 transmembrane-juxtamembrane domain (SEZ6L2 tm jm) comprises an amino acid sequence that is at least about 95%, 96%, 97%, 98%, 99% or 100% identical to the amino acid sequence represented by SEQ ID NO.: 66,
the EGFP comprises an amino acid sequence that is at least about 95%, 96%, 97%, 98%, 99% or 100% identical to the amino acid sequence represented by SEQ ID NO.: 67, and
the P2A comprises an amino acid sequence that is at least about 95%, 96%, 97%, 98%, 99% or 100% identical to the amino acid sequence represented by SEQ ID NO. 68.

9. A nucleic acid comprising a polynucleotide encoding the CAR of claim 1.

10. A vector comprising a polynucleotide encoding the CAR of claim 1.

11. A cell comprising the CAR of claim 1.

12. A composition comprising the cell of claim 11.

13. A method of treating disease in a subject in need thereof, comprising administering to the subject an effective amount of the cell of claim 11;
wherein preferably, the disease is ROR1 positive cancer; more preferably, the cancer is selected from one or more of blood cancer and solid cancer, wherein preferably, the cancer includes, but is not limited to, gastric cancer, pancreatic cancer, esophageal cancer, lung cancer, ovarian cancer, head and neck cancer, bladder cancer, cervical cancer, sarcoma, cytoma, colon cancer, kidney cancer, colorectal cancer, liver cancer, melanoma, breast cancer, myeloma, neuroglioma, skin cancer, adrenal cancer, uterine cancer, testicular cancer, prostate cancer, blood cancer, leukemia and/or lymphoma.

14. A method of treating both ROR1 and CD19 positive cancer, comprising administering to the subject the dual CAR of claim 5;
wherein preferably, the cancer is selected from one or more of blood cancer and solid cancer, wherein preferably, the cancer includes, but is not limited to, gastric cancer, pancreatic cancer, esophageal cancer, lung cancer, ovarian cancer, head and neck cancer, bladder cancer, cervical cancer, sarcoma, cytoma, colon cancer, kidney cancer, colorectal cancer, liver cancer, melanoma, breast cancer, myeloma, neuroglioma, skin cancer, adrenal cancer, uterine cancer, testicular cancer, prostate cancer, blood cancer, leukemia, and/or lymphoma.

15. A method of producing a CAR-T cell comprising:
(1) introducing to a host cell the nucleic acid of claim 9, and
(2) isolating and/or expanding the CAR-T cells following the introduction.

16. A nucleic acid comprising a polynucleotide encoding the dual CAR of claim 5.

17. A vector comprising a polynucleotide encoding the dual CAR of claim 5.

18. A cell comprising the dual CAR of claim 5.

19. A composition comprising the cell of claim 18.

20. A method of treating disease in a subject in need thereof, comprising administering to the subject an effective amount of the cell of claim 18;
wherein preferably, the disease is ROR1 positive cancer; more preferably, the cancer is selected from one or more of blood cancer and solid cancer, wherein preferably, the cancer includes, but is not limited to, gastric cancer, pancreatic cancer, esophageal cancer, lung cancer, ovarian cancer, head and neck cancer, bladder cancer, cervical cancer, sarcoma, cytoma, colon cancer, kidney cancer, colorectal cancer, liver cancer, melanoma, breast cancer, myeloma, neuroglioma, skin cancer, adrenal cancer, uterine cancer, testicular cancer, prostate cancer, blood cancer, leukemia and/or lymphoma.
